SEQUENCE LISTING (16 sequences)

SEQ ID NO: 1 - DNA, 48 bp, Artificial Sequence
  Description of Artificial Sequence: Synthetic oligonucleotide
  modified_base (31)..(48): a, c, t, g, unknown or other
ttttttttttttttttttttttttttttttnnnnnnnnnnnnnnnnnn

SEQ ID NO: 2 - DNA, 30 bp, Artificial Sequence
  Description of Artificial Sequence: Synthetic oligonucleotide
  Description of Combined DNA/RNA Molecule: Synthetic oligonucleotide
aagcagtggtatcaacgcagagtgaatggg

SEQ ID NO: 3 - DNA, 23 bp, Artificial Sequence
  Description of Artificial Sequence: Synthetic oligonucleotide
aagcagtggtatcaacgcagagt

SEQ ID NO: 4 - DNA, 54 bp, Artificial Sequence
  Description of Artificial Sequence: Synthetic oligonucleotide
  modified_base (35)..(54): a, c, t, g, unknown or other
gtctcgtgggctcggagatgtgtataagagacagnnnnnnnnnnnnnnnnnnnn

SEQ ID NO: 5 - DNA, 25 bp, Artificial Sequence
  Description of Artificial Sequence: Synthetic oligonucleotide
aagcagtggtatcaacgcagagtac

SEQ ID NO: 6 - DNA, 66 bp, Artificial Sequence
  Description of Artificial Sequence: Synthetic oligonucleotide
aatgatacggcgaccaccgagatctacacgcctgtccgcggaagcagtggtatcaacgcagagtac

SEQ ID NO: 7 - DNA, 37 bp, Artificial Sequence
  Description of Artificial Sequence: Synthetic primer
gcctgtccgcggaagcagtggtatcaacgcagagtac

SEQ ID NO: 8-9 - skipped (2 entries)

SEQ ID NO: 10 - DNA, 24 bp, Artificial Sequence
  Description of Artificial Sequence: Synthetic oligonucleotide
tatttctactgcgacgagtaggag

SEQ ID NO: 11 - DNA, 24 bp, Artificial Sequence
  Description of Artificial Sequence: Synthetic oligonucleotide
tatttctactgcgacgaggaggag

SEQ ID NO: 12 - DNA, 25 bp, Artificial Sequence
  Description of Artificial Sequence: Synthetic oligonucleotide
acgtgagcccctcgggctgattcgt

SEQ ID NO: 13 - DNA, 25 bp, Artificial Sequence
  Description of Artificial Sequence: Synthetic oligonucleotide
acgtgagcccctcgggcggcttcgt

SEQ ID NO: 14 - DNA, 20 bp, Artificial Sequence
  Description of Artificial Sequence: Synthetic oligonucleotide
  modified_base (1)..(20): a, c, t, g, unknown or other
nnnnnnnnnnnnnnnnnnnn

SEQ ID NO: 15 - DNA, 25 bp, Artificial Sequence
  Description of Artificial Sequence: Synthetic oligonucleotide
aagcagtggtatcaacgcagagtac

SEQ ID NO: 16 - RNA, 10 nt, Artificial Sequence
  Description of Artificial Sequence: Synthetic oligonucleotide
ucgucuagau

(19) United States
(12) Patent Application Publication
(10) Pub. No.: US 2023/0304069 A1
(43) Pub. Date: Sep. 28, 2023

(54) METHODS AND COMPOSITIONS FOR SINGLE CELL ANALYSIS

(71) Applicant: The Johns Hopkins University, Baltimore, MD (US)

(72) Inventors: Claire Marie Bell, Crownsville, MD (US); Donald Zack, Baltimore, MD (US); Tza-Huei Wang, Baltimore, MD (US); Aniruddha Kaushik, BAltimore, MD (US); Pengfei Zhang, Baltimore, MD (US)

(21) Appl. No.: 18/020,340

(22) PCT Filed: Aug. 12, 2021
(86) PCT No.: PCT/US2021/045802

§ 371 Date: Feb. 8, 2023

Related U.S. Application Data
(60) Provisional application No. 63/065,433, filed on Aug. 13, 2020.

(51) Int. Cl. C12Q 1/6806; C12Q 1/6853; C12Q 1/686

(52) U.S. Cl. CPC C12Q 1/6806; C12Q 1/6853; C12Q 1/686; C12Q 2600/16

(57) ABSTRACT

Provided herein are methods and compositions for simultaneously analyzing DNA and RNA from the same cell using sequencing methodologies. Methods and compositions provided herein are useful for cell characterization at the transcriptome and genomic levels, cell screening, and lineage tracing, for example. Also provided herein are kits for simultaneously analyzing DNA and RNA.

### CROSS-REFERENCE TO RELATED APPLICATIONS

This application claims benefit of priority under 35 U.S.C. § 119(e) of U.S. Provisional Application No. 63/065,433, filed Aug. 13, 2020. The disclosure of the prior application is considered part of and is herein incorporated by reference in the disclosure of this application in its entirety.

### BACKGROUND OF THE INVENTION

### Field of the Invention

The present invention relates generally to single cell analysis and more specifically to simultaneous analysis of DNA and RNA from the same cell.

### Background Information

Single cell transcriptomic sequencing (scRNA-seq) has shown that individual cells are unique, heterogeneous units with their own subtle but importantly different gene expression profiles. Separately, single-cell DNA sequencing has highlighted genetic heterogeneity in multicellular organisms and its role in inherited diseases, cancer, and aging. Genomic sequences and transcriptomic profiles each independently reflect intercellular differences and coupling targeted genomic information to the transcriptome of a single cell can provide a high resolution to the direct and diverse relationship between genotype and phenotype.

The ability to simultaneously assay genomic DNA (gDNA) and messenger RNA (mRNA) from the same single cell is important for attributing differential transcriptomic output to specific cell genotypes. A high-throughput method for single cell measurements of mRNA and DNA can provide advantages to multiple applications, ranging from clinical diagnostics to CRISPR screens and cellular barcoding. Existing approaches for generating this particular combination of data are limited by time consumption and low throughput. More recent high-throughput methodologies exist that employ cDNA as a proxy for genomic sequence, but these restrict the measurable DNA sequence to only expressed regions of the genome. Thus, there exists a need for efficient and cost-effective methods of simultaneously analyzing DNA and RNA from the same cell.

### SUMMARY OF THE INVENTION

The present invention is based on the seminal discovery that DNA and RNA from the same single cell can be captured and analyzed simultaneously using sequencing methodologies.

While the illustrative examples herein provide DNA and RNA analysis in droplets, it is understood that the methods of the invention can be performed in non-droplet cell encapsulation methods as well, including for example, Fluorescence activated cell sorting (FACS), gravity, microfluidics and the like.

In some embodiments, provided herein are methods of simultaneously analyzing DNA and RNA from the same cell including: (a) providing a droplet including a single cell, wherein the single cell is lysed providing nucleic acid in the droplet; (b) performing a first polymerase chain reaction (PCR) reaction on DNA in the droplet, thereby generating amplicons including a 3′ poly(dA) sequence and a bead oligonucleotide sequence; (c) capturing the RNA on a microparticle comprising a bead oligonucleotide sequence; (d) breaking the droplet and separating the supernatant comprising the amplicons from the RNA captured on the microparticle; and (e) performing a reverse transcription reaction transcribing the RNA. In one aspect, methods provided herein further include preparing libraries of the separated amplicons and transcribed RNA. In another aspect, methods provided herein further include performing a PCR reaction on

the transcribed RNA. In another aspect, the methods provided herein further comprising enzymatically modifying the amplicons. In an additional aspect, the amplicons are modified by a lambda nuclease and a terminal transferase. In a further aspect, the methods provided herein further comprise biotinylating the amplicons by biotinylated second strand synthesis. In some aspects, the methods provided herein further comprise subjecting the amplicons to mung bean nuclease modification. In another aspect, the methods provided herein further include performing a second PCR reaction on the amplicons. In an additional aspect, the methods provided herein further include performing a third PCR reaction on the amplicons. In certain aspects, the forward primers for the second PCT reaction on the amplicons include sites for sequencing. In a further aspect, the forward and reverse primers in the third PCR reaction on the amplicons include sites for sequencing. In yet another aspect, methods provided herein further include sequencing the transcribed RNA and amplicons after the third PCR reaction. In one aspect the microparticle that captures RNA is a bead. In another aspect, the bead includes bead oligonucleotide sequences. In yet another aspect, the bead oligonucleotide sequences include a barcode. In a further aspect, the barcode includes a cellular barcode and a unique molecular identifier. In certain aspects, the oligonucleotide sequences further include a poly(dT) sequence. In some aspects, the oligonucleotide sequences further include a PCR handle for reverse transcription and PCR. In certain aspects, methods provided herein further include mapping sequences of separated molecules that include a matching cellular barcode to the same cell. In one aspect, the DNA analyzed by the methods provided herein is genomic DNA, mitochondrial DNA, or a combination thereof. In another aspect, the RNA analyzed by the methods provided herein is messenger RNA (mRNA), long non-coding RNA, or a combination thereof. In a further aspect, first PCR reverse primers include a poly(dT) sequence. In yet a further aspect, the reverse transcription reaction includes Moloney Murine Leukemia Virus (M-MLV)-reverse transcriptase and template switching oligonucleotides.

In some embodiments, provided herein are methods of simultaneously analyzing DNA and RNA from the same cell including: (a) providing a droplet including a single cell, wherein the single cell is lysed providing nucleic acid in the droplet; (b) performing a first polymerase chain reaction (PCR) reaction on DNA in the droplet, thereby generating amplicons including a 3′ poly(dA) sequence and a bead oligo nucleotide sequence; (c) capturing the RNA with a microparticle comprising a bead oligonucleotide sequence; (d) breaking the droplets and separating the supernatant containing the amplicons from the RNA captured on the microparticle; (e) performing a reverse transcription reaction on the RNA including the bead oligonucleotide sequence; (f) enzymatically modifying the amplicons and performing a second PCR reaction on the amplicons; and (g) performing a PCR reaction on the transcribed RNA. In one aspect, methods provided herein further include performing a third PCR reaction on the amplicons.

In some embodiments, provided herein are methods of analyzing a transcriptome of a genome-edited cell including: (a) determining a genotype of a single cell by sequencing amplicons prepared by methods of simultaneously analyzing DNA and RNA from the same cell provided herein, thereby identifying edited and unedited cells; (b) sequencing transcribed RNA prepared by methods of simultaneously analyzing DNA and RNA from the same cell provided herein; (c) mapping sequences of amplicons and sequences of transcribed RNA that include a matching cellular barcode to the same cell; and (d) grouping sequences of amplicons and sequences of transcribed RNA from edited and unedited cells according to matching genome edits. In another aspect, methods provided herein include preparing a sequencing library of amplicons and preparing a sequencing library of transcribed RNA before sequencing transcribed molecules. In a further aspect, single cells include a genomic barcode. In yet a further aspect, edited cells include one or more mutations in the genomic barcode.

In some embodiments, provided herein are methods of determining tumor heterogeneity that include simultaneously analyzing DNA and RNA from a tumor cell using any of the methods provided herein.

In some embodiments, provided herein are methods of determining somatic mosaicism that include simultaneously analyzing DNA and RNA from a cell using any of the methods provided herein. In one aspect, the cell is a normal cell. In another aspect, the cell is a disease cell. In yet another aspect, the disease cell is a tumor cell. In a further aspect, somatic mosaicism includes a mutation or a chromosomal rearrangement.

In some embodiments, provided herein are methods of screening for perturbations in cells modified with guide RNAs that include simultaneously analyzing DNA and RNA of a cell in a population of modified cells using any of the methods provided herein. In one aspect, the cells are modified with a library (e.g., lentiviral) of guide RNAs representative of a range of genes. A readout of integrated guide RNAs provides information as to perturbed genes. In one aspect, the cells are modified with a gene modifying agent selected from a CRISPR-associated (Cas) protein, a Cre DNA recombinase, a TALEN, a zinc finger nuclease, a homing endonuclease, or a targeted SPO11 nuclease.

In some embodiments, provided herein are methods of probing genetic thresholds on a phenotype that include simultaneously analyzing DNA and RNA from a cell using any of the methods provided herein. In one aspect, the phenotype is a normal phenotype. In another aspect, the phenotype is a disease phenotype.

In some embodiments, provided herein are methods of genotyping cells that include simultaneously analyzing DNA and RNA from a cell using any of the methods provided herein. In one aspect, the cell is a tumor cell, a genome-edited cell, a disease cell, or a normal cell.

In some embodiments, provided herein are methods of tracing a lineage of a cell that include simultaneously analyzing DNA and RNA from the cell the lineage of which is being traced using any of the methods provided herein. In one aspect, methods of tracing cell lineage provided herein further include marking the cell with a barcode. In another aspect the barcode is a DNA barcode. In yet another aspect, the DNA barcode is an editable DNA barcode.

In some embodiments, provided herein are oligonucleotides that include a PCR handle for reverse transcription and PCR, a barcode, and a poly(dT) sequence. In one aspect, the barcode includes a cellular barcode and a unique molecular identifier. In another aspect, the oligonucleotides are attached to a microparticle. In a further aspect, the microparticle includes a bead. In yet a further aspect, oligonucleotides provided herein further include an amplified DNA sequence including a 3′ poly(dA) sequence.

In another embodiment a kit including a combination lysis and PCR buffer comprising a lysis component, PCR components, and a reaction buffer, wherein the lysis component comprises a nonionic, non-denaturing detergent, wherein the PCR component comprises a polymerase, deoxynucleoside triphosphates, and PCR primers, and wherein the reaction buffer comprises MgCl2, Tween-80, carrier protein, Tris and NaCl is provided. In a specific aspect, the Tris buffer is at pH 8.0. In some aspects, the reaction buffer comprises dimethyl sulfoxide (DMSO). In one aspect, the kit further includes droplet generation oil; and a microparticle. In one aspect, the kit further includes instructions to generate aqueous solution-in-oil droplets comprising a cell and a microparticle. In another aspect, the kit further includes a polymerase and instructions to perform a polymerase chain reaction (PCR) reaction on DNA in the droplet. In one aspect, the PCR reaction generates amplicons comprising a 3′ poly(dA) sequence and bead oligonucleotide sequence. In another aspect, the kit further includes a reverse transcriptase and instructions to perform a reverse transcription reaction on the RNA. In a further aspect, the kit also includes instructions to separate the amplicons and the RNA prior to performing the reverse transcriptase reaction on the RNA. In some aspects, the combination lysis and PCR buffer includes a lysis component, PCR components, and a reaction buffer. In various aspects, the lysis component includes Igepal CA-630. In many aspects, the PCR components include a polymerase, deoxynucleoside triphosphates, and PCR primers. In various aspects, the polymerase is a Q5 Hot Start High-Fidelity DNA polymerase, Phusion polymerase or a KOD polymerase. In many aspects, the carrier protein is BSA or ubiquitin. In some aspects, the reaction buffer includes MgCl₂, Tween-80, a carrier protein, Tris and NaCl.

### BRIEF DESCRIPTION OF THE DRAWINGS

FIG. 1 shows examples of multiple “omes” measured from the same single cells.

FIG. 2 shows applications and challenges of reading DNA and RNA from the same cell.

FIG. 3 shows an overview of the DREAM-seq (DNA/RNA Extraction, Amplification, and Multiplexing) method for simultaneous analysis of DNA and RNA from single cells.

FIG. 4 shows barcoding of DNA amplicons during the droplet PCR.

FIG. 5 shows an overview of the serial PCR for generating droplet amplicons and turning them into sequencing libraries.

FIG. 6 shows droplets before PCR.

FIG. 7 shows droplets after PCR.

FIG. 8 shows an overview of the enzymatic processing of the droplet amplicons.

FIG. 9 shows direct PCR in droplets.

FIG. 10 shows the addition of a poly(dA) sequence to amplicons.

FIG. 11 shows the incorporation of bead oligonucleotide sequences into amplicons during droplet PCR.

FIG. 12 shows droplet amplicons with bead oligonucleotide sequences enriched after the second PCR.

FIG. 13 shows advantages of in-droplet genotyping of edited cells.

FIG. 14 shows possible editing outcomes of CRISPR/Cas9 editing of pluripotency factors in stem cells.

FIG. 15 shows sequencing of genomic and transcriptomic data from the same cell following CRISPR/Cas9 editing that targeted c-Myc or KLF4.

FIGS. 16A and 16B shows advantages and limitations of techniques for studying cell differentiation and development. FIG. 16A shows single cell trajectory algorithms. FIG. 16B shows lineage-tracing DNA barcodes.

FIG. 17 shows that combining single cell RNA sequencing (scRNA-seq) and lineage tracing overcomes limitations of each technique.

FIG. 18 shows methods of developing high resolution maps of tissue differentiation using capture of mRNA and lineage barcodes.

FIG. 19 shows cells identified as mouse or human based on their captured transcripts.

FIG. 20 shows cells identified as mouse or human based on their gDNA amplicons.

FIG. 21 shows whether cells with human or mouse transcripts had more human or mouse associated amplicons.

FIG. 22 shows the species determination based on transcripts and amplicons for a set of cells.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Provided herein, in some illustrative embodiments, are methods of simultaneously analyzing DNA and RNA from the same cell that include (a) providing a droplet including a single cell, wherein the single cell is lysed providing nucleic acid in the droplet; (b) performing a first polymerase chain reaction (PCR) reaction on DNA in the droplet, thereby generating amplicons including a 3′ poly(dA) sequence and a bead oligonucleotide sequence; (c) capturing the RNA on a microparticle containing a bead oligonucleotide sequence; (d) breaking the droplet and separating the supernatant containing the amplicons from the RNA captured on the microparticles; and (e) performing a reverse transcription reaction on the RNA including the bead oligonucleotide sequence, thereby transcribing the RNA.

Also provided herein, in some embodiments, are methods of simultaneously analyzing DNA and RNA from the same cell that include (a) providing a droplet including a single cell, wherein the single cell is lysed providing nucleic acid in the droplet; (b) performing a first polymerase chain reaction (PCR) reaction on DNA in the droplet, thereby generating amplicons including a 3′ poly(dA) sequence and a bead oligonucleotide sequence; (c) capturing the RNA on a microparticle containing a bead oligonucleotide sequence; (d) breaking the droplet and separating the supernatant containing the amplicons from the captured RNA; (e) performing a reverse transcription reaction on the RNA including the bead oligonucleotide sequence, thereby transcribing the RNA; (f) enzymatically modifying the amplicons and performing a second PCR reaction on the modified amplicons; and (g) performing a second PCR reaction on the transcribed RNA, thereby amplifying transcribed RNA.

Methods of simultaneously analyzing DNA and RNA from the same cell provided herein include providing droplets that include nucleic acid from a single cell. Droplets encompass single cells that are lysed within the droplets, thus releasing nucleic acid into the droplets. As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, mitochondrial DNA, chloroplast DNA, plasmid DNA, cDNA, cell-free DNA(cfDNA), mRNA, tRNA, rRNA, siRNA, micro RNA (miRNA or miR), hnRNA, and long non-coding RNA. As used herein, the term “nucleic acid molecule” is meant to include fragments of nucleic acid molecules as well as any full-length or non-fragmented nucleic acid molecule, for example.

Any DNA and RNA can be analyzed using the methods provided herein. In one aspect, DNA analyzed using the methods provided herein is genomic DNA, mitochondrial DNA, or a combination thereof. In another aspect, the RNA is messenger RNA (mRNA), long non-coding RNA, or a combination thereof.

Methods of simultaneously analyzing DNA and RNA from the same cell provided herein include performing a first polymerase chain reaction (PCR) reaction on DNA in the droplets. A first PCR on DNA in the droplets generates amplicons that include a 3′ poly(dA) sequence and a bead oligonucleotide sequence. The 3′ poly(dA) sequence of amplicons derived from DNA can be used to prime the bead oligonucleotides and incorporate their sequences into the amplicons. Accordingly, in one aspect, first PCR reverse primers include a poly(dT) sequence. The poly(dT) sequence included in first PCR reverse primers results in the introduction of the 3′ poly(dA) sequence of amplicons derived from DNA, such as genomic DNA, mitochondrial DNA, or a combination thereof. In another aspect, a bead reverse primer can be used that binds to the bead oligo sequences that any amplicons have incorporated, allowing them to be further amplified in the subsequent cycles of the first PCR. The bead oligonucleotide sequence can be used to match to the RNA with the same bead oligonucleotide sequence.

Methods of simultaneously analyzing DNA and RNA from the same cell provided herein include capturing the RNA. In one aspect, the RNA are captured on a support. Any suitable support can be used to capture the RNA, such as a microparticle, for example. In some aspects, the microparticle for capture of amplicons and RNA is a bead. In a further aspect, the beads for capture of the RNA include oligonucleotide sequences. Oligonucleotide sequences can be attached to the bead by any suitable method, including covalent and non-covalent interactions. In one aspect, oligonucleotides are covalently attached to the bead.

In another aspect, bead oligonucleotide sequences include a barcode. Barcodes can be used to identify the origin or source of a nucleic acid molecule or of a nucleic acid sequence. A barcode can include any number of nucleotides. As an example, a barcode can include about 10 to about 35 nucleotides. As another example, a barcode can include about 12 to about 25 nucleotides. As yet another example, a barcode can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, or more nucleotides. As yet another example, a barcode can include at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, or more nucleotides. In yet another aspect, the barcode includes a cellular barcode and a unique molecular identifier (UMI). Cellular barcodes can be used to identify the cell a nucleic acid molecule came from and allow for grouping of sequence reads generated from nucleic acid molecules into cell categories. Thus, sequence reads of nucleic acid molecules from the same cell can be grouped together. UMIs can be used to identify a nucleic acid molecule that gave rise to a sequence read. Accordingly, UMIs can be used to group together sequence reads generated from the same nucleic acid molecule.

In a further aspect, oligonucleotide sequences include a poly(dT) sequence. A poly(dT) sequence allows for the capture and tagging of nucleic acid molecules that include a poly(dA) sequence. Exemplary molecules that can interact with an oligonucleotide poly(dT) sequence include mRNA, long non-coding RNA, and amplicons derived from DNA with poly(dA) sequences generated by PCR. In yet a further aspect, oligonucleotide sequences include a PCR handle for reverse transcription and PCR. The PCR handle can be used for reverse transcription and/or PCR of captured nucleic acid molecules.

Methods of simultaneously analyzing DNA and RNA from the same cell provided herein further include breaking the droplets that include nucleic acid from a single cell and separating the supernatant containing the amplicons from the captured RNA. Further, the method provides performing a reverse transcription reaction on the captured RNA, thereby transcribing the RNA. In one aspect, the transcription reaction is performed on beads after capture of RNA and breaking of the droplets. In another aspect, the reverse transcription reaction is performed using Moloney Murine Leukemia Virus (M-MLV)-reverse transcriptase and template switching oligonucleotides. In yet another aspect, transcribing the amplicons and the RNA incorporates microparticle oligonucleotide sequences into transcribed molecules.

Methods of simultaneously analyzing DNA and RNA from the same cell provided herein further include separating amplicons and captured RNA. In one aspect, a PCR reaction is performed on the transcribed RNA. Any suitable method can be used for separating transcribed molecules. In one aspect, the amplicons are enzymatically modified. The certain aspects the amplicons are enzymatically modified using a lambda nuclease to prevent the reverse strand from acting as a downstream template amplicon without a bead oligonucleotide sequence. In some aspects, the amplicons are further enzymatically modified using a terminal transferase and ddNTPs to prevent forward strands without a bead oligonucleotide sequence from priming reverse strands with a bead oligonucleotide sequences inducing template switching. In a further aspect, the amplicons are then biotinylated by a biotinylated second strand synthesis reaction. In some aspects, the amplicons are subjected to mung bean nuclease modification. In some aspects, the amplicons are removed from the supernatant using streptavidin beads. In certain aspects, a second PCR reaction is performed on the amplicons using forward primers including sequencing sites. Primers that target regions of interest in transcribed amplicons can be used in the PCR reactions. Any region or sequence of transcribed amplicons can be a region of interest and be targeted by primers for amplification. More than one primer pair or more than one set of primers can be used to amplify regions of interest.

. In yet another aspect, forward primers for PCR on tagged amplicons include sites for sequencing. Accordingly, a PCR reaction on tagged amplicons using primers can be performed before preparing sequencing libraries and sequencing transcribed molecules.

Methods of simultaneously analyzing DNA and RNA provided herein further include preparing libraries of separated amplicons and transcribed RNA. Any suitable method for library preparation can be used. In one aspect, libraries of transcribed RNA (i.e., cDNA sequencing libraries) are prepared using tagmentation methods followed by amplification. In another aspect, amplicon sequencing libraries are prepared from the supernatant derived from breaking the droplet and subjected to enzymatic modification using lambda nuclease and terminal transferase with ddNTPs followed by being subjected to a biotinylated second strand synthesis reaction, mung bean nuclease modification, and selection of biotinylated molecules with streptavidin beads followed by two rounds of PCR, as detailed above.

Methods of simultaneously analyzing DNA and RNA from the same cell provided herein further include sequencing the separated transcribed molecules. Any sequencing method can be used, including Sanger sequencing using labeled terminators or primers and gel separation in slab or capillary systems, and Next Generation Sequencing (NGS), for example. Exemplary NGS methodologies include the Roche 454 sequencer, Life Technologies SOLiD systems, the Life Technologies Ion Torrent, BGI/MGI systems, Genapsys systems, and Illumina systems such as the Illumina Genome Analyzer II, Illumina MiSeq, Illumina HiSeq, Illumina NextSeq, and Illumina NovaSeq instruments. In one aspect, methods of simultaneously analyzing DNA and RNA from the same cell provided herein further include mapping sequences of separated transcribed molecules having a matching cellular barcode to the same cell.

In some embodiments, provided herein are methods of analyzing a transcriptome of a genome-edited cell that include a) determining a genotype of a single cell by sequencing transcribed amplicons prepared by any of the methods provided herein that include simultaneously analyzing DNA and RNA from the same cell, thereby identifying edited and unedited cells; (b) sequencing transcribed RNA prepared by any of the methods provided herein that include simultaneously analyzing DNA and RNA from the same cell; (c) mapping sequences of transcribed amplicons and sequences of transcribed RNA that include a matching cellular barcode to the same cell; and (d) grouping sequences of transcribed amplicons and sequences of transcribed RNA from edited and unedited cells according to matching genome edits.

As used herein, the term “transcriptome” means all RNA transcripts in a cell or in a population of cells. Accordingly, RNA transcripts include coding and non-coding RNA, and the term “transcriptome” encompasses both coding and non-coding RNA, unless context clearly indicates otherwise. As used herein, the term “genome editing” means insertion, deletion, modification, or replacement of DNA in the genome of a cell or organism. Any type of genetic engineering can be used for genome editing, including gene targeting, conditional gene targeting, homologous recombination, and use of nucleases, such as meganucleases, zinc finger nucleases (ZFNs), transcription activator-like effector nucleases (TALENs), and the CRISPR/Cas system. As used herein, the term “edited cell” means a cell whose genome includes a desired insertion, deletion, modification, or replacement of genomic DNA. The genome of an edited cell may include a mutation as a result of editing. Alternatively, editing can be used to correct a mutation, i.e., restore a mutation or alteration to wild-type or to an equivalent of wild-type. As used herein, “unedited cell” means a cell whose genome does not include a desired insertion, deletion, modification, or replacement of genomic DNA. Accordingly, the genome of an unedited cell may be wild-type or include a mutation, based on the nature or purpose of genome editing.

Methods of analyzing a transcriptome of a genome-edited cell provided herein include performing a first polymerase chain reaction (PCR) reaction on DNA in a droplet, thereby generating amplicons including a 3′ poly(dA) sequence. Generation of amplicons that include a poly(dA) sequence allows the amplicons to be tagged by a support that includes an oligonucleotide sequence, such as a bead that includes an oligonucleotide sequence having a poly(dT) sequence. Accordingly, in one aspect, first PCR reverse primers include a poly(dT) sequence.

In one aspect, single cells in the methods of analyzing a transcriptome of a genome-edited cell provided herein include a genomic barcode. In another aspect, edited cells of the methods provided herein include one or more mutations in the genomic barcode. In yet another aspect, mutations in the genomic barcode result from genome editing. In one aspect, the barcodes are introduced by genome editing. In other aspects, the barcodes are introduced, for example by methods that cause random or untargeted genomic changes (e.g., use of transposases or lentiviruses).

In one aspect, methods of analyzing a transcriptome of a genome-edited cell provided herein include separating the supernatant containing amplicons from the captured RNA. The captured RNA is subjected to reverse transcriptase reaction transcribing the RNA. The isolated amplicons are subjected enzymatic modification as described previously and further PCR reactions. In another aspect, methods of analyzing a transcriptome of a genome-edited cell further include preparing a sequencing library of transcribed amplicons and preparing a sequencing library of transcribed RNA before sequencing transcribed molecules. Any of the methods provided herein can be used for preparing sequencing libraries, including tagmentation methods for cDNA sequencing library preparation and PCR. In one aspect, the amplicons are enzymatically modified using lambda nuclease and a terminal transferase followed by a biotinylated second strand synthesis reaction and modification by mung bean nuclease prior to a second and third PCR reaction.

Methods of analyzing a transcriptome of a genome-edited cell provided herein include capturing and transcribed RNA. In one aspect, transcribed RNAs are captured on a support, such as a microparticle, for example. In another aspect, the microparticle is a bead. In yet another aspect, beads for capture of transcribed RNA include oligonucleotide sequences. In another aspect, bead oligonucleotide sequences include a barcode that includes a cellular barcode and a unique molecular identifier (UMI). In a further aspect, bead oligonucleotide sequences further include a poly(dT) sequence, a PCR handle for reverse transcription and PCR, or any combination thereof. Any suitable reverse transcriptase, including engineered reverse transcriptase, can be used to transcribe captured RNA. In one aspect, the reverse transcriptase is Moloney Murine Leukemia Virus (M-MLV)-reverse transcriptase, using template switching oligonucleotides.

Any type of nucleic acid can be analyzed using the methods of analyzing a transcriptome of a genome-edited cell provided herein. In one aspect, a first PCR reaction is performed on DNA in droplets as provided herein, wherein the DNA is genomic DNA, mitochondrial DNA, or a combination thereof. In another aspect, transcribed and/or captured RNA includes mRNA, long non-coding RNA, or a combination thereof.

Any of the methods provided herein can be used to determine tumor heterogeneity, for example. Methods provided herein can also be used to determine somatic mosaicism. In one aspect, single cells analyzed by the methods provided herein for determining somatic mosaicism are normal cells. In another aspect, single cells analyzed by the methods provided herein for determining somatic mosaicism are tumor cells. In certain aspects, somatic mosaicism includes a mutation or a chromosomal rearrangement.

Methods provided herein can also be used for screening for perturbations in cells modified with guide RNAs in a population of modified cells. In one aspect, the cells are modified with a library (e.g., lentiviral) of guide RNAs representative of a range of genes. Any suitable type of library can be used to generate genome edits. A readout of integrated guide RNAs provides information as to perturbed genes. In one aspect, the cells are modified using a gene modifying agent selected from a CRISPR-associated (Cas) protein, a Cre DNA recombinase, a TALEN, a zinc finger nuclease, a homing endonuclease, or a targeted SPO11 nuclease.

Methods provided herein can be used for probing genetic thresholds on phenotype. In one aspect, the phenotype is a normal phenotype. In another aspect, the phenotype is a disease phenotype. Accordingly, methods provided herein can be used to determine the number and/or type of genetic markers or genetic changes that contribute to a phenotype of interest.

Any of the methods for simultaneously analyzing DNA and RNA from a cell provided herein can be used to genotype cells. In one aspect, the cell is a tumor cell. In another aspect, the cell is a genome-edited cell. In a further aspect, the cell is a disease cell. In yet a further aspect, the cell is a normal cell. Any of these cell types are optionally barcoded cells. Accordingly, the genotype of any cell can be determined using the methods provided herein. Exemplary cells that can be analyzed include single cells from any organ, single cells from any cell culture, primary cells, cells that have been preserved by any suitable method, including single frozen cells, single formalin-fixed cells, methanol fixed, or single cells from formalin-fixed paraffin-embedded (FFPE) tissue, by way of example.

Methods provided herein for simultaneously analyzing DNA and RNA from the same cell can be used for tracing the lineage of the cell. In one aspect, the cell whose lineage is being traced is marked with a barcode. In another aspect, the barcode is a DNA barcode. In a further aspect, the DNA barcode is an editable barcode. Any of the genome-editing methods provided herein can be used to edit a DNA barcode, including use of a gene modifying agent such as a CRISPR-associated (Cas) protein, a Cre DNA recombinase, a TALEN, a zinc finger nuclease, a homing endonuclease, or a targeted SPO11 nuclease, for example.

Provided herein, in some embodiments, are oligonucleotides including a PCR handle for reverse transcription, a barcode, and a poly(dT) sequence. In one aspect, the barcode includes a cellular barcode and a unique molecular identifier (UMI). In yet another aspect, the oligonucleotides are attached to microparticles, such as beads, for example. In a further aspect, oligonucleotides provided herein include tagging DNA with a 3′ poly(dA) sequence and/or a bead oligonucleotide sequence. In yet a further aspect, the DNA that includes a 3′ poly(dA) sequence is genomic DNA, mitochondrial DNA, or a combination thereof.

Generally, oligonucleotides provided herein are single-stranded. Oligonucleotides can be of any length. For example, oligonucleotides can have a length of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 60 nucleotides, 70 nucleotides, 80 nucleotides, 90 nucleotides, 100 nucleotides, 125 nucleotides, 150 nucleotides, 200 nucleotides, or more nucleotides, and any number or range in between. PCR handles, barcodes, including cellular barcodes and UMIs, and poly(dT) sequences can be arranged in any order and be separated by any number of nucleotides or be contiguous, i.e., be located next to each other without other nucleotides in between. Cellular barcodes and UMIs included in barcodes of oligonucleotides provided herein can be contiguous, i.e., located next to each other, or located apart from each other. For example, cellular barcodes can be separated by 1 nucleotide, 2 nucleotides, nucleotides, 3 nucleotides, 4 nucleotides, 5 nucleotides, 6 nucleotides, 7 nucleotides, 8 nucleotides, nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, or more nucleotides. Cellular barcodes, UMIs, poly(dT) sequences, and PCR handles can be of any length, including 1 nucleotide, 2 nucleotides, nucleotides, 3 nucleotides, 4 nucleotides, 5 nucleotides, 6 nucleotides, 7 nucleotides, 8 nucleotides, nucleotides, 9 nucleotides, 10 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotide, 50 nucleotides, or more nucleotides, and any number or range in between.

In another embodiment, a kit including a combination lysis and PCR buffer; droplet generation oil; and a microparticle is provided. In one aspect, the combination lysis and PCR buffer includes a lysis component, PCR components, and a reaction buffer. In another aspect, the lysis component includes Igepal CA-630. In one aspect, the PCR components include a polymerase, deoxynucleoside triphosphates and PCR primers. In various aspects, the polymerase is a Q5 Hot Start High-Fidelity DNA polymerase. In another aspect, the reaction buffer includes MgCl₂, Tween-80, a carrier protein Tris and NaCl. In many aspects, the carrier protein is BSA or ubiquitin.

In one aspect, the kit further includes instructions to generate aqueous solution-in-oil droplets comprising a cell and a microparticle.

In another aspect, the kit further includes a polymerase and instructions to perform a polymerase chain reaction (PCR) reaction on DNA in the droplet. In some aspects, the PCR reaction generates amplicons comprising a 3′ poly(dA) sequence.

In one aspect, the kit further includes a reverse transcriptase and instructions to perform a reverse transcription reaction on RNA. In various aspects, the reverse transcription reaction includes Moloney Murine Leukemia Virus (M-MLV)-reverse transcriptase and template switching oligonucleotides.

In other aspects, the kit further includes instructions to separate the supernatant containing the amplicons from the RNA prior to performing the reverse transcriptase reaction the RNA.

In some aspects, the microparticle includes a bead. In one aspect, the bead includes oligonucleotide sequences. In many aspects, the oligonucleotide sequences include a barcode. In some aspects, the barcode includes a cellular barcode and a unique molecular identifier. In one aspect, the oligonucleotide sequences further include a poly(dT) sequence. In other aspects, the oligonucleotide sequences further include a PCR handle for reverse transcription and PCR.

The following examples are provided to further illustrate the embodiments of the present invention but are not intended to limit the scope of the invention. While they are typical of those that might be used, other procedures, methodologies, or techniques known to those skilled in the art may alternatively be used.

### Example 1

This example illustrates design of a method for analysis of DNA and RNA from the same cell.

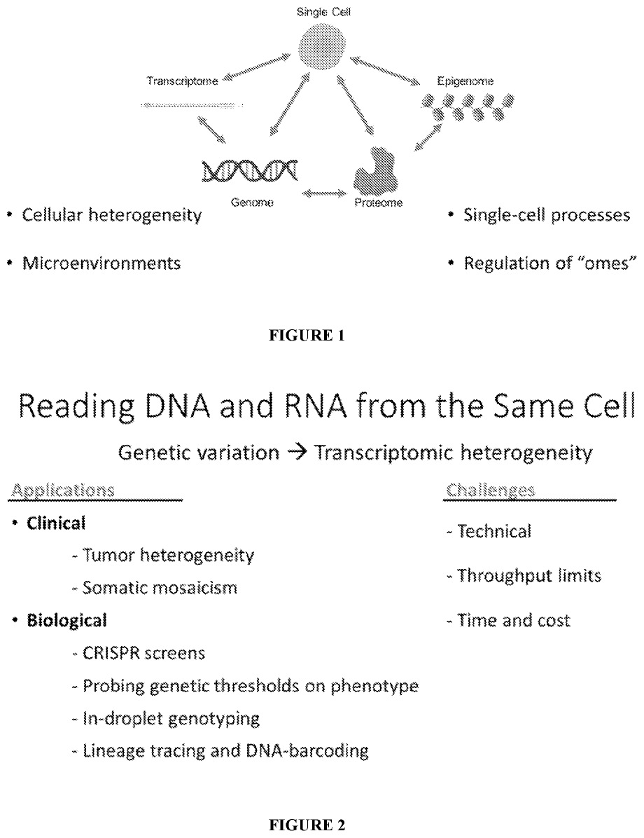

Collecting information from multiple “omes” from the same single cells allows for a more refined analysis and understanding of cellular heterogeneity and cellular function, as shown in FIG. 1, for example. In addition, reading DNA and RNA from single cells allows for direct association of genetic variation with transcriptomic heterogeneity. Exemplary applications for reading DNA and RNA from the same cell are shown in FIG. 2. Understanding tumor heterogeneity and genome-transcriptome relationships allows for selection of patient-specific treatments, including for patients with somatic mosaicism resulting from disease mutations or chromosomal rearrangements, for example. A targeted genomic readout of transcriptomic data is also useful for CRISPR screens and other transgene integration screens. It can eliminate batch effects in single cell RNA-seq runs, if there is no need to keep samples separate, and it can pair cellular genomic barcoding techniques such as lineage tracing with single cell transcriptomics, for example. However, protocols aimed at capturing and processing RNA are not suitable for DNA, and challenges such as low cellular throughput and time-intensive and expensing protocols have hampered development of methods for simultaneous analysis of RNA and DNA from the same cell.

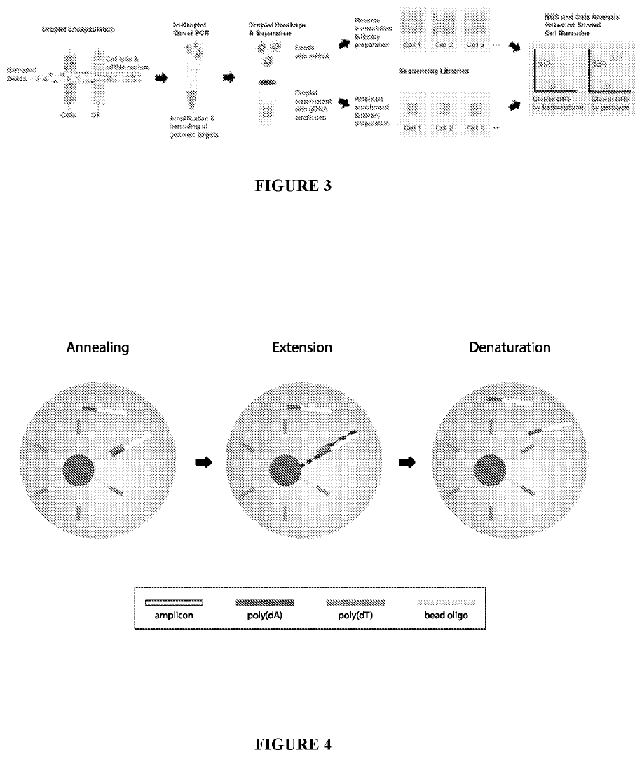

To overcome these limitations, a system that adapts 3′ poly(dA) capture-based scRNA-seq methods to simultaneously barcode both targeted DNA and RNA from single cells was developed using a microfluidics-based platform (FIGS. 3 and 4). DREAM-seq (DNA/RNA Extraction, Amplification, and Multiplexing) allows for analyzing targeted DNA sequences and mRNA expression from single cells. DREAM-seq derives its output directly from genomic DNA (gDNA), allowing any amplifiable region of the genome—translated or untranslated—to be associated with a transcriptome. DREAM-seq was developed by combining microfluidic droplet-based single-cell and bead encapsulation with droplet PCR to create a high-throughput technique for collecting integrated single cell genomic and transcriptomic data. As cells are compartmentalized in droplets, they lyse, releasing both their mRNA and gDNA into the solution. Each droplet's contents undergo their own isolated amplification reaction, generating compartmentalized, cell-specific genomic amplicons that are tagged with the microbead's oligonucleotide sequences. At the same time, mRNA from the lysed cells is captured by the same microbead oligonucleotides. As shown in FIG. 4, beads capturing amplified gDNA and mRNA are coated with oligonucleotides, each containing a bead-specific cell barcode, a unique molecular identifier (UMI), and a poly(dT) sequence.

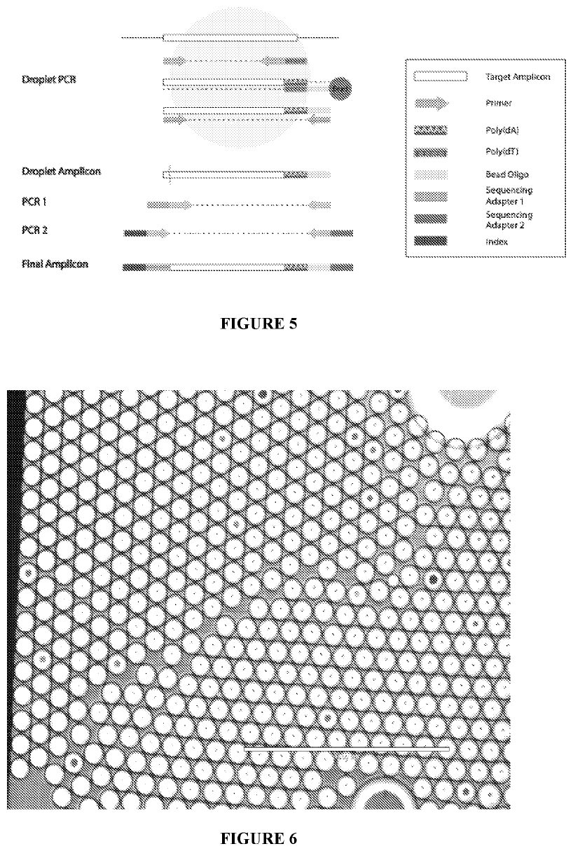

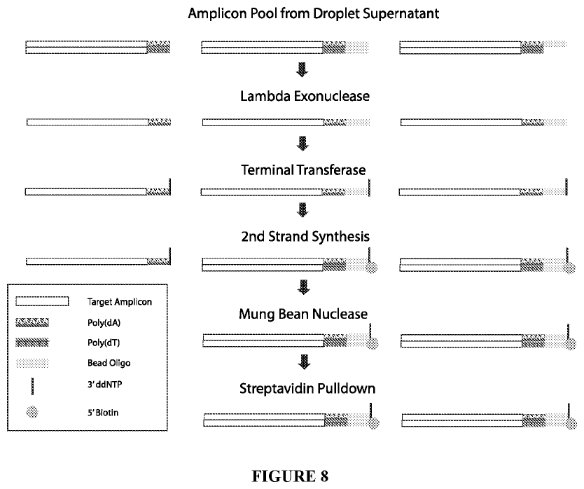

DREAM-seq is performed as follows (FIG. 3): A combination lysis and PCR buffer is flowed through a microfluidic device with Drop-seq beads, cells, and oil, generating 0.8˜1 nL-sized droplets (FIG. 6). After cells and beads are captured, cells are lysed and PCR is performed within each droplet, amplifying target regions of the genome and barcoding them with microbead oligonucleotide sequences (FIGS. 4 and 5). In addition to genomic DNA, target regions from mitochondrial DNA can be amplified as well. The droplets are broken open, and the droplet supernatant containing barcoded amplicons is separated from the captured mRNA immobilized on the beads. The mRNA on the beads undergoes reverse transcription, incorporating the bead oligonucleotide sequences, and the cDNA is amplified and prepared as a sequencing library. Separately, the droplet supernatant undergoes targeted enzymatic modifications and two additional serial PCR steps to prepare an amplicon sequencing library (FIGS. 5 and 8). After sequencing the libraries, amplicons and cDNA sequences originating from the same cell are remarried during analysis by their shared cell barcodes. Thus, a shared bead barcode allows for assignment of sequences to the same cell, for example.

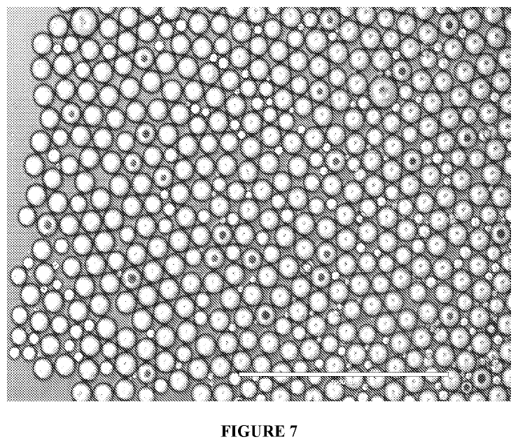

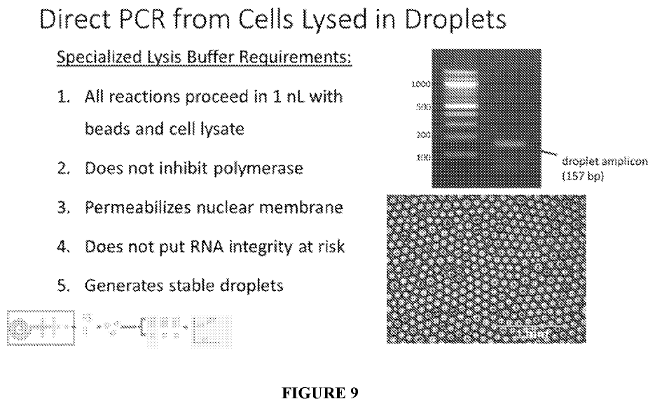

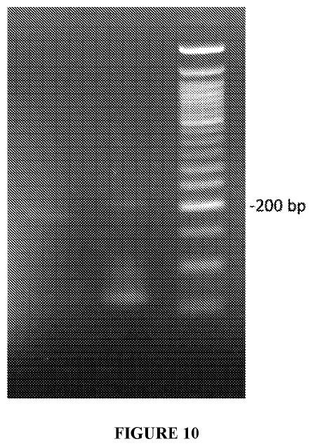

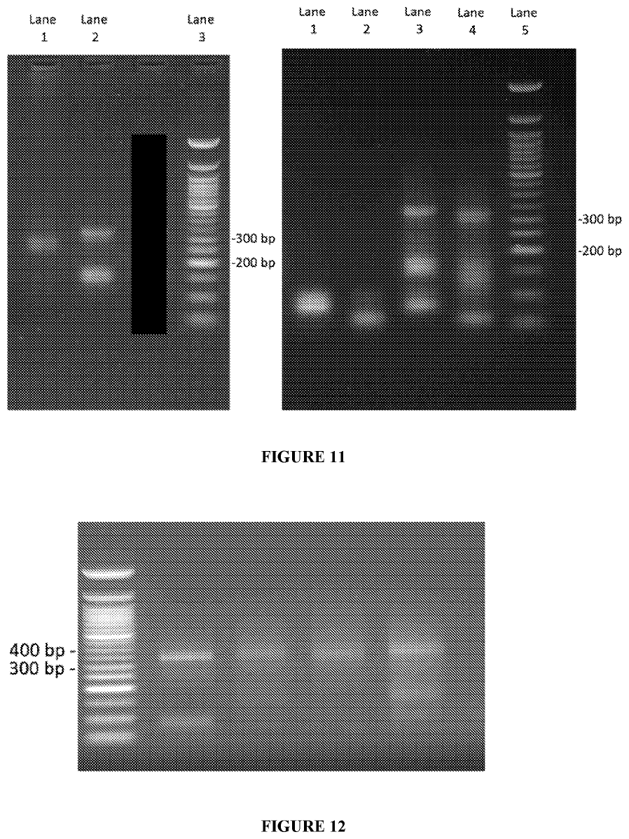

DREAM-seq relies on PCR amplification of genomic targets directly from cells lysed after droplet encapsulation. This requires overcoming PCR inhibition introduced by the limited reaction volume, beads, and cell lysis. A buffer was developed to effectively lyse cells and proceed directly to PCR in droplets (FIG. 9). Importantly, this buffer was also capable of generating stable, consistently-sized droplets that remained intact during PCR (FIGS. 6 and 7). Primers were then used to amplify target regions of gDNA and append a 3′ poly(dA) tract to the PCR products (FIGS. 5 and 10), and a subset of forward amplicon strands annealed with the poly(dT) sequences of the bead oligonucleotides during the droplet PCR. This effectively primed the transcription of the rest of the oligonucleotide barcode sequence, including the cell barcode, and incorporated it into the 3′ end of the target amplicon. During the denaturation step of the PCR, these barcoded strands were be released back into the droplet supernatant, where they could be retrieved separately from the mRNA and enriched with further PCR (FIGS. 5, 11, and 12). The two types of molecules, DNA amplicons in the droplet supernatant and mRNA captured on the beads, were physically separated and individually prepared as libraries and sequenced. Genotyping data and transcriptomic information were bioinformatically re-merged by the bead-derived cell barcodes incorporated into the respective transcripts.

Before creating sequencing libraries from gDNA-derived amplicons, droplets were broken open using a filter and supernatant containing the amplicons was purified and concentrated. The purified supernatant was then subjected to a series of enzymatic reactions (FIG. 8) to prevent molecules that had not incorporated a bead oligonucleotide sequence from unintentionally priming molecules that did incorporate one. Without removing or rendering inert these shorter amplicons, they contribute to wide-scale template switching in the subsequent PCR reactions, incorporating random bead oligo sequences into themselves, and preventing originally cell-specific amplicons from being faithfully identified. The purified droplet supernatant was first digested by lambda exonuclease which degrades DNA from the 5′ to 3′ direction and eliminated reverse strands of the amplicons. Terminal transferase was then used to add dideoxy nucleosides to the 3′ ends of the remaining amplicon strands to prevent them from unintentional priming and extension. Afterwards, Taq polymerase and a biotinylated bead oligonucleotide-specific primer were used to synthesize biotinylated reverse strands off of the amplicons that had originally incorporated a bead oligonucleotide. Lastly, persisting single stranded amplicons without bead oligonucleotide sequences were digested with mung bean nuclease. The remaining amplicons were purified using streptavidin-coated beads and proceeded into the library preparation reactions.

To create sequencing libraries for the DNA amplicons, the molecules bound to streptavidin-coated beads were subjected to a second round of PCR using a nested forward primer to append the first part of the sequencing adapters (FIG. 4, middle). The PCR products were gel extracted, and then used as template for a third PCR to generate the final amplicon sequencing library (FIG. 4, bottom). cDNA libraries were generated using standard methods of tagmentation to create shorter fragments with sequencing adapters, followed by a final amplification to generate sequencing libraries.

### Example 2

This example shows an exemplary protocol for performing simultaneous DNA and RNA analysis from the same cell.

DREAM-Seq (DNA/RNA Extraction, Amplification, and Multiplexing) Stepwise Protocol:

Equipment:

Real-time PCR detection machine

Thermocycler capable of ramping temperatures (preferably with the ability to hold PCR plates)

E-Gel Power Snap Electrophoresis Device (Thermo Fisher)

2100 Bioanalyzer (Agilent)

Qubit fluorometer (Thermo Fisher)

Tube rotator

Magnetic bead rack

Drop-seq setup (i.e. pumps, microscope, chips, etc. See mccarrolllab.org/dropseq/for instructions on building one)

Fuchs-Rosenthal Hemocytometer

MiSeq (Illumina)

TABLE 1
Materials and Reagents:

| Item | Supplier | Part # |
|---|---|---|
| Agilent High Sensitivity DNA Kit | Agilent | 5067-4626 |
| AMPure XP beads | Beckman Coulter | A63880 |
| BD Syringes, 50 mL | Fisher | 13-689-8 |
| Cell strainers (35 μm) | Fisher/Falcon | 352235 |
| Cell strainers (100 μm) | Corning | 352360 |
| Cryostor CS10¹ | Stemcell Technologies | 07930 |
| DNA Clean and Concentrator-5 | Zymo Research | D4013 |
| ddNTP Set, Sequencing Grade | Millipore Sigma | 03732738001 |
| dNTPs 100 mM | Thermo Fisher | R0181 |
| Drop-seq beads | ChemGenes | MACOSKO-2011-10(V+) |
| Dynabeads MyOne Streptavidin C1 | Thermo Fisher | 65001 |
| 0.5M EDTA, pH 8.0 | Corning | 46-034-CI |
| E-Gel 50 bp DNA Ladder | Thermo Fisher | 10488099 |
| E-Gel EX Agarose Gels, 2% | Thermo Fisher | G401002 |
| E-Gel SizeSelect II Agarose Gels, 2% | Thermo Fisher | G661012 |
| E-Gel Sizing DNA Ladder | Thermo Fisher | 10488100 |
| Eppendorf DNA LoBind Microcentrifuge Tubes | Fisher | 13-698-790 |
| Ethanol | Sigma-Aldrich | E7023 |
| Exonuclease I | New England Biolabs | M0293L |
| Ficoll 400 | Sigma-Aldrich | F4375 |
| HBSS (no calcium, no magnesium, no phenol red)² | Gibco | 14175095 |
| Igepal CA-630 | Sigma-Aldrich | I8896 |
| KAPA HiFi HotStart ReadyMix | Roche | KK2602 |
| Lambda Exonuclease | New England Biolabs | M0262S |
| Magnesium Chloride, 1M | Quality Biological | 351-033-721 |
| Maxima H Minus Reverse Transcriptase | Thermo Fisher | EP0753 |
| Mineral Oil | Sigma-Aldrich | M5904 |
| Monarch DNA Gel Extraction Kit | New England Biolabs | T1020S |
| Mung Bean Nuclease | New England Biolabs | M0250S |
| Nextera XT DNA Library Preparation Kit | Illumina | FC-131-1024 |
| Nuclease-free water | Quality Biological | 351-029-131 |
| NxGen RNAse Inhibitor | Lucigen | 30281-2 |
| PBS, pH 7.4 (no calcium, no magnesium, no phenol red)² | Gibco | 10010023 |
| PCR plates³ | Sarstedt | 72.1979.102 |
| PDMS DropSeq chip | FlowJEM | |
| Phusion Hot Start Flex DNA Polymerase⁴ | New England Biolabs | M0535L |
| Q5 Hot Start High-Fidelity DNA Polymerase⁴ | New England Biolabs | M0493L |
| Qubit dsDNA HS Assay Kit | Invitrogen | Q32854 |
| QX200 Droplet Generation Oil for EvaGreen | Bio-Rad | 1864006 |
| RNaseZap | Thermo Fisher | AM9780 |
| Sodium Chloride, 5M | Quality Biological | 351-036-101 |
| Sodium dodecyl sulfate | Sigma-Aldrich | 436143 |
| SSC Buffer, 20× Concentrate | Sigma-Aldrich | SRE0068-1L |
| SYBR Green I nucleic acid gel stain | Invitrogen | S7563 |
| Taq DNA Polymerase, recombinant | Thermo Fisher | EP0401 |
| Terminal Transferase | New England Biolabs | M0315S |
| Tris HCl, 1M pH 8.0 | Quality Biological | 351-007-101 |
| Tween 20 | Sigma-Aldrich | P1379 |
| Tween 80 | Sigma-Aldrich | P1754 |
| Uberstrainers, 5 μm | Pluriselect | 43-70005-03 |
| Ubiquitin from bovine erythrocytes | MilliporeSigma | U6253-25MG |
| UltraPure BSA (5%) | ThermoFisher | AM2616 |

¹CS10 freezing medium can be used to freeze single cell suspensions for DREAM-seq. For frozen cells that are particularly buoyant, samples may be frozen at high concentration in CS10 and diluted directly (at least 1:20) with no negative effects on the reactions. Other freezing medias have not been tested for their effects on in-droplet cell lysis/PCR.
²Both PBS and HBSS have been successfully tested as cell suspension buffers, either may be used.
³PCR plates or tubes used for droplet PCR must be emulsion safe to prevent droplets from merging.
⁴Both polymerases have been successfully tested and used in the direct droplet PCR reaction. Other polymerases may be substituted as well, although it should be noted that Taq-based polymerases do not work in this system. It is important that any polymerase used for droplet PCR is a hot start polymerase.

Buffer Formulations and Reaction Mixes:

2× DREAM-seq direct PCR/lysis buffer (1 mL):¹
50 ul 1M Tris pH 8.0
100 ul 10% Igepal ca-630
30 ul 5M NaCl
100 ul 20% Tween-80
92 ul 5% Ubiquitin (50 mg/mL in H₂O)
40 ul 300 mM MgCl₂
30 ul polymerase (60 units)
80 ul 10 mM dNTP mix (diluted in H₂O)
100 ul primer mix²
378 ul nuclease-free H₂O
¹DREAM-seq buffer may be frozen and stored at −20° C. for later use, with or without primers included.
²2% DMSO (1% final in droplets) enhances the PCR in some cases and may be tested with each individual primer set.
³Final droplet primer concentrations of 0.4 μM forward, 0.1 μM reverse, and 0.4 μM Bead_R are favorable for promoting interaction of the amplicons with the bead oligos. This corresponds to a mix of 8 μM forward and Bead_R, and 2 μM reverse primers being added to the buffer formulation.

TE-SDS
10 mM Tris pH 8.0
1 mM EDTA
0.5% SDS

TE-TW
10 mM Tris pH 8.0
1 mM EDTA
0.01% Tween-20

PBS (or HBSS)-BSA
PBS/HBSS
0.025% UltraPure BSA³

2× BW-Tween
10 mM Tris-HCl pH 7.5
1 mM EDTA
2 M NaCl
0.2% Tween-20
³If using an alternative BSA, make sure that there is no EDTA in the formulation.

TABLE 2
Primers Sequences:

| | |
|---|---|
| ¹DREAM-seq_F | N*N*N*N*N*NNNNNNNNNNNNNNN |
| | SEQ ID NO: 14 |
| | (Genomic target/experiment specific) |
| ¹DREAM- | /5Phos/TTTTTTTTTTTTTTTTTTTTTTTTTTTTTTNNNNNNNNNN |
| seq_R_polyT | NNNNNNNN |
| | SEQ ID NO: 1 (Genomic target/experiment specific) |
| TSO | AAGCAGTGGTATCAACGCAGAGTGAATrGrGrG |
| | SEQ ID NO: 2 |
| SMART | AAGCAGTGGTATCAACGCAGAGT |
| | SEQ ID NO: 3 |
| Biotin_Bead_R | /5Biosg/AAGCAGTGGTATCAACGCAGAG+T+A+C |
| | SEQ ID NO: 15 |
| XT_Nested_F | GTCTCGTGGGCTCGGAGATGTGTATAAGAGACAGNNNNNNN |
| | NNNNNNNNNNNNN SEQ ID NO: 4 |
| | (Genomic target/experiment specific) |
| Bead_R | AAGCAGTGGTATCAACGCAGAG+T+A+C |
| | SEQ ID NO: 5 |
| Nextera XT i7 | support- |
| Indexes | docs.illumina.com/SHARE/AdapterSeq/Content/SHARE/AdapterSeq/ |
| | Nextera/DNAIndexesNXT.htm |
| P5-SMART | AATGATACGGCGACCACCGAGATCTACACGCCTGTCCGCGG |
| | AAGCAGTGGTATCAACGCAGAG*T*A*C |
| | SEQ ID NO: 6 |
| Custom Read 1 | GCCTGTCCGCGGAAGCAGTGGTATCAACGCAGAGTAC |
| Primer | SEQ ID NO: 7 |

¹It is extremely important that DREAM-seq primers are PAGE-purified.

Designing and Testing Primers for DREAM-Sea:

It is critical to test primers designed for DREAM-seq before performing any runs. DREAM-seq primers with shorter products (100-300 bp) amplify more efficiently and are preferred. When beads are present in the reaction, ideal primers produce clean bands with no visible primer dimers. Primer sets should be tested with the poly(T) tract already incorporated in the reverse primer.

Testing DREAM-seq primers:
Dissociate single cells and resuspend in PBS/BSA at a concentration of 2×10\hat{ }⁶ cells/mL
Spin down 15,000 drop-seq beads in a PCR tube and carefully remove the supernatant Resuspend beads in 7.5 ul of direct PCR/lysis buffer (Note: it is critical to add primers to the buffer before resuspending the beads) (Note: Primer mix should include Bead_R primer.)
Add 7.5 ul of cells to the beads/buffer reaction, and pipette to mix
Run the following PCR program:
   95° C. 2:30
   For 35 cycles:
   98° C. 0:05
   58° C. 0:10
   68° C. 0:15 (longer may be needed if amplicon is over 400 bp)
Purify the reaction with a Zymo spin column and 5×binding buffer (there is no need to remove beads before adding binding buffer)
Run the PCR product out on a 2% agarose gel to visualize

After initial DREAM-seq primer testing, XT_Nested_F primers need to be tested and the whole serial PCR optimized. The 5′ portion of these primers adds the first part of the Nextera sequencing adapter to the amplicons, while the 3′ portion binds specifically to the amplicon. XT_Nested_F primers should bind at least 15 bases downstream of the DREAM-seq_F sequence. The product from this adapter PCR reaction should be clean with no visible off-target amplification. Nested primers and serial PCR can be tested and optimized in bulk by modeling the droplet reactions. Supernatant can be mixed from separate bulk per reactions that represent the distribution of empty droplets, droplets with only cells, droplets with only beads, and droplets with a cell and a bead. The mixed supernatant can then be used as a template for testing XT_Nested_F primers in a qPCR reaction, and the resulting curves will determine the number of droplet PCR cycles and adapter PCR cycles will be needed for actual DREAM-seq experiments.

XT_Nested_F Primer Testing and Optimization:
Dissociate single cells and resuspend in PBS/BSA at a concentration of 2×10\hat{ }⁶ cells/mL
Prepare reactions with equal volumes PBS/BSA or cells and buffer for the following conditions:
   PBS/BSA only−16×20 ul rxns
   PBS/BSA only+20 k beads−2×20 ul rxn
   Cells only−2×20 ul rxn
   Cells+15 k beads−2×15 ul rxn
Run the following PCR with 1 reaction of each condition for 35 cycles to visualize on a gel as controls, and the remaining reactions for 28 cycles to mix and test supernatant:
   95° C. 2:30
   For 28/35 cycles:
   98° C. 0:05
   58° C. 0:10
   68° C. 0:15 (longer may be needed if amplicon is over 400 bp)
Spin down the 28 cycle PCR reactions, and mix together the following volumes of supernatant, while avoiding aspirating any beads for a total of 320 ul of mixed supernatant:
   289 ul PBS/BSA only (90.25% of mixed supernatant)
   15.2 ul PBS/BSA+beads (4.75% of mixed supernatant)
   15.2 ul Cells only (4.75% of mixed supernatant)
   0.8 ul Cells+beads (0.25% of mixed supernatant)
Purify the mixed supernatant with a Zymo spin column and 5×binding buffer, eluting in a final volume of 40 ul H₂O.
Prepare the following qPCR mix:
   12.5 ul KAPA HiFi HotStart Readymix
   2.5 ul 10× SYBR Green I (diluted in H₂O)
   0.75 ul 10 uM XT_Nested_F
   0.75 ul 10 uM Bead_R
   3.5 ul H₂O
   5 ul purified mixed supernatant
Aliquot 3×5 ul for qPCR. Save the remaining 10 ul for PCR.
Run the following qPCR/PCR:
   95° C. for 3:00
   For 40 cycles (qPCR) or 35 cycles (PCR):
   98° C. for 0:10
   60° C. for 0:20
   72° C. for 0:30
[Plate Read] (qPCR only)
Purify the 35 cycle PCR reactions from step 3 with Zymo columns
Run the PCR products from steps 8 (no need for purification) and 9 on a 2% agarose gel (Note: If the Cq values from step 8 are ≥30, run the qPCR product on the gel instead of the PCR product, to ensure that it can be seen)

After verifying that the adapter PCR product is clean, use the qPCR results to determine the number of droplet PCR cycles and adapter PCR Cycles for your experiment. If the qPCR reaches early exponential phase before 20 cycles, consider decreasing the amount of droplet PCR cycles to no lower than 25 for actual experiments. If the qPCR reaches early exponential phase after 25 cycles, consider increasing the amount of droplet PCR cycles (cDNA quality has not been tested at higher than 32 cycles). The number of adapter PCR cycles should be determined based on how many cycles it takes for the reaction to reach early-to-mid exponential phase.

Running DREAM-Sea:

Bead/Buffer Preparation:
Thaw or make fresh the necessary volume of direct PCR/lysis buffer for the experiment.
Remove 2×10 ul for positive and negative PCR controls. Controls may be prepared with genomic DNA and PBS/BSA.
For every 1 mL of buffer, spin down 120,000 Drop-seq beads at 1,000×g for 1 minute.
Carefully remove supernatant.
Resuspend beads in direct PCR/lysis buffer. Keep on ice until the run.

Cell Preparation:
Resuspend dissociated and pelleted cells in PBS or HBSS+0.025% BSA and pass through a 35 μm cell strainer. Count cells.
Dilute cells to 120,000/mL in PBS or HBSS+0.025% BSA.
Proceed with cell encapsulation. NOTE: Adjust flow rates ahead of time to ensure that droplets are >0.8 nL (˜118 μm diameter).

Direct PCR in Droplets:
Use a P1000 pipette to remove excess oil from the bottom of the droplet collection tube. Press the pipette down to its first stop, then bring the tip to the bottom of the tube and press to the second stop to expel any droplets that may have entered the tube. Wait a few seconds for the droplets to float up, and then release the pipette to aspirate the excess oil.
Aliquot 50 ul of droplets per reaction into high-profile PCR tubes or plates.
Add 50 ul of mineral oil on top of each PCR reaction. Make sure that the mineral oil is fully covering the droplets. Seal or cap the tubes/plates.
Note the number of PCR reactions for estimating supernatant volume later.
Run the following PCR program (it is important to ramp temperature changes to preserve droplet integrity):
   95° C. 2:30 (Ramp 2° C./s)
   For X cycles (optimized before experiment):
   98° C. 0:05 (Ramp 2° C./s)
   58° C. 0:10 (Ramp 2° C./s)
   68° C. 0:15 (Ramp 2° C./s)
   Then:
   4° C. for ∞ (Ramp 2° C./s)

Tip—begin preparing the RT mix (below) during the droplet PCR.

Same-Day Bead Processing:
Remove the screw cap and white luer-lok tip from an uberstrainer unit, and place the uberstrainer (yellow) into a 50 mL conical, and re-pool the droplets into the uberstrainer.
Raise the plunger of a 50 mL syringe to fill the syringe with air, and attach the syringe to the uberstrainer using the luer-lok screw cap.
Push the plunger into the syringe, using the air to break the droplets and filter the droplet supernatant into the 50 mL conical.
Unscrew the syringe, and repeat with 10-20 additional mLs of air to push through any remaining supernatant.
Transfer the uberstrainer and screw cap to a new 50 mL conical tube and save the first conical with the supernatant. This supernatant contains your barcoded amplicons, and can be stored at 4° C. until further processing.
Remove the plunger fully from the syringe, and attach the syringe once more to the screw cap.
Add 30 mL 6× SSC buffer to the syringe and use the plunger to push the buffer and residual air through the strainer to wash the beads.
Remove the syringe and unscrew the screw cap from the filter.
Place the screw cap luer-lok up on a flat surface. Insert a P1000 pipette tip by hand into the opening of the luer-lok screw, and push until a plastic disk is removed from inside the screw cap.—Discard the disk.
Screw the white luer-lok tip back onto the screw cap, and place the cap tip-down into a 1.5 mL centrifuge tube rack.
Insert the yellow uberstrainer upright into the cap, and press lightly until a vacuum seal forms.
Add 1 mL of 6×SSC buffer into the uberstrainer, pipette up and down several times to kick up the beads, and then transfer the beads and buffer to a 1.5 ml DNA LoBind microcentrifuge tube.
Add an additional 0.5 mL of 6×SSC buffer to the uberstrainer to kick up and wash out any remaining beads, and transfer them to the same microcentrifuge tube.
Centrifuge the beads for 1 minute at 1000×g and carefully remove the supernatant.
Wash the beads once with ˜300 ul 5×Maxima RT Buffer.
Resuspend the beads in the following RT mix:
   RT Mix (200 ul, enough for ˜90 k beads):
      75 ul H2O
      40 ul Maxima 5× RT Buffer
      40 ul 20% Ficoll PM-400
      20 ul 10 mM dNTPs
      5 ul RNase Inhibitor
      10 ul 50 uM TSO primer
      10 ul Maxima H-RTase (add just before use)
Incubate the beads at room temperature for 30 minutes, with rotation.
Incubate the beads at 42° C. for 90 minutes, with rotation.
Wash the beads once with 1 mL TE-SDS, then twice with 1 mL TE-TW.
Beads can be stored at 4° C. in TE-TW until exonuclease.

Exonuclease:
Wash beads once with 1 mL 10 mM Tris pH 8.0
Resuspend beads in the following exonuclease mix:
   Exonuclease Mix (200 ul, enough for ˜90 k beads):
      20 ul Exo I Buffer
      170 ul H₂O
      10 ul Exol
Incubate the beads at 37° C. for 50 minutes with rotation.
Wash the beads once with 1 mL TE-SDS, then twice with 1 mL TE-TW.
Beads can be stored at 4° C. in TE-TW until exonuclease until PCR.

Total cDNA Amplification:

Tip: After counting the beads (step 1 below), it may be useful to optimize the number of PCR cycles needed for each experiment before amplifying cDNA from the total pool of beads. Optimization can be done by following the below steps with one or two aliquots of beads run for different numbers of cycles and analyzing the cDNA, or by adding sybr green to the PCR reaction and performing qPCR to identify how many cycles are required to reach early-to-mid exponential phase.
Count the number of beads per sample using a hemocytometer. Tip: As the beads can be difficult to distribute evenly, it can be easier to dilute them, load 20 ul onto the hemocytometer, count the entire number of beads and then divide by 20 ul to get the diluted value of beads per ul.
Wash the beads once with 1 mL H2O.
Resuspend beads in the following PCR mix (want to make 50 ul per 5000 beads):
   24.6 ul H₂O
   0.4 ul 100 uM SMART PCR primer
   25 ul 2×Kapa Hifi
Aliquot 50 ul of beads/PCR mix per PCR tube. Make sure to keep the beads well-mixed while pipetting.
Run the following PCR program:
   95° C. 3 min
   For 4 cycles:
   98° C. 20 s
   65° C. 45 s
   72° C. 3 min
   For 8-16 cycles (should be optimized):
   98° C. 20 s
   67° C. 20 s
   72° C. 3 min
   Then:
   75° C. 5 min
   4° C. ∞

Total cDNA Purification and Analysis:

Occasionally in DREAM-seq, particularly in experiments that require higher numbers of droplet PCR cycles, the total cDNA yields a wider or bimodal size distribution, possibly because of RNA degradation during the PCR. When this occurs, the shorter fragments tend to bind more efficiently to the sequencing flow cell and are largely comprised of polyA, so it is important to remove them during purification.

It was found that doing two sequential 0.6× Ampure bead purifications (following the manufacturer's instructions) was sufficient if the cDNA is uncompromised and yields total cDNA with an average size of 1200-1500 bp and a smooth, normal distribution, as assessed on a Bioanalyzer. With this protocol, final purified cDNA was eluted in ˜6-8 ul per original PCR reaction.

However, if a lower size distribution of cDNA is observed, it is recommended to purify the total cDNA by concentrating it with Zymo clean and concentrator columns, running it out on a 2% E-gel EX agarose gel, and gel-extracting fragments larger than 500 bp (may or may not be visible on the gel). One well of the gel was loaded for every 50 PCR rxns, to avoid overloading. It was found that the NEB Monarch gel extraction kit combined with the E-gel EX provided very favorable cDNA yield. With this protocol, final purified cDNA was eluted in ˜1-2ul per original PCR reaction.

Purified total cDNA should be assessed on a Bioanalyzer High Sensitivity DNA chip, according to the manufacturer's instructions. Total cDNA should be at least 150 pg/ul.

cDNA Library Preparation and Purification:
Preheat a thermocycler to 55° C.
In a PCR tube, add 600 pg of total cDNA and bring to a volume of 5 ul with H2O.
Add 10 ul of Nextera Tagment DNA buffer and mix by pipetting.
Add 5 ul of Nextera Amplicon Tagment Mix and mix by pipetting. Spin down.
Incubate the reaction at 55° C. for 5 minutes.
Add 5 ul of Nextera Neutralization Buffer. Mix by pipetting and spin down.
Incubate the reaction at room temperature for 5 minutes.
On ice, add the following reagents to the PCR tube:
   15 ul Nextera PCR Master Mix
   8 ul H₂O
   1 ul 10 uM P5-SMART primer
   1 ul 10 uM N70× index primer
Vortex and spin down, then run the following PCR program:
   73° C. 3 min
   95° C. 30 s
   For 12-15 cycles:
   95° C. 10 s
   55° C. 30 s
   72° C. 30 s
   Then:
   72° C. 5 min
   4° C. ∞
Purify the library using 0.6× Ampure beads as per the manufacturer's instructions.
Elute in 50 ul H2O and repeat step 10.
Elute the purified library in 10 ul H₂O.
Assess the library on a BioAnalyzer High Sensitivity DNA chip, as per the manufacturer's instructions.

The final library should average between 500-800 bp, with an ideal concentration of 4 nM or greater. Libraries can be stored at 4° C. or −20° C. before sequencing.

Droplet Supernatant/Amplicon Purification:
Supernatant collected during the post-PCR droplet breakage and filtering steps should have been stored at 4° C. before processing.
Estimate the volume of droplet supernatant, discounting any volume from droplet or mineral oil. A ballpark estimation of 1 mL supernatant per 25 droplet PCR reactions may be used.
Add 5 volumes worth of Zymo clean and concentrate DNA Binding Buffer to the supernatant.
Vortex to mix.
Calculate the number of Zymo clean and concentrate spin columns to use. One spin column should be used for every 4 mL of supernatant+binding buffer.
Purify the mixture using the spin columns as per the manufacturer's instructions. The buffer/supernatant mixture can be added to the spin column 800 ul at a time, up to 5 times per spin column, before washing and eluting. When adding the mixture to the spin columns, try to add only the middle aqueous phase and avoid oil/protein from the top and bottom of the tube.
Elute in 6 ul H2O per spin column.
Purified droplet supernatant can be stored at 4° C. or −20° C.

Lambda Exonuclease Digestion:

This reaction removes the reverse strands of amplicons, which is necessary to minimize extreme template switching in the subsequent PCR steps.
1. Mix together the following 10 ul reaction:
   1 ul 10× Lambda Exonuclease Reaction Buffer
   0.3 ul Lambda Exonuclease
   8.7 ul purified droplet supernatant
2. Incubate at 37° C. for 45 minutes.
3. Heat inactivate at 80° C. for 15 minutes.
4. Move directly into the following TDT blocking reaction.

Terminal Transferase 3′ End Blocking: This reaction blocks the 3′ end of the amplicons, preventing shorter fragments without incorporated bead oligo sequences from priming longer ones in the subsequent PCR reactions.
1. Add the following reaction components to the heat-inactivated lambda exonuclease reaction from the previous step:
   7.5 ul Terminal Transferase Reaction Buffer
   1.5 ul 1 mM ddNTPs
   7.5 ul 2.5 mM CoCl₂
   6 ul Terminal Transferase
   43.5 ul H₂O
2. Incubate at 37° C. for 2 hours.
3. Heat inactivate at 75° C. for 20 minutes.
4. Clean up the reaction using a Zymo column and a 7:1 ratio of binding buffer (DNA amplicons should be mostly single stranded at this point).
5. Elute amplicons in 10 ul H2O.

Biotin labelling of tagged amplicons
1. Add the following reaction components to the eluted amplicon from the previous step:
   2 ul 10× Taq Buffer with KCl
   0.5 ul 10 mM dNTPs
   2.4 ul 25 mM MgCl2
   5 ul 20 uM Biotin_Bead_R
   0.2 ul Taq DNA Polymerase
2. Incubate for 2 minutes at 95° C., then 45 minutes at 63° C.
3. Purify with a Zymo column (5× binding buffer) and elute in 6 ul H2O.

Single Stranded DNA Digestion and Streptaviding Pulldown
1. Add the following reaction components to the eluted amplicon from the previous step:
   1 ul 10× Mung Bean Nuclease buffer
   1 ul Mung Bean Nuclease
   3 ul H₂O
2. Incubate for 1 hour at 30° C.
3. Add 0.2 ul of 0.5% SDS to inactivate the nuclease.
4. Mix streptavidin Dynabeads well by vortexing 30 seconds.
5. Pipette 10 ul Dynabeads into a PCR tube.
6. Using a magnetic bead rack, wash the Dynabeads 3× with 100 ul 1× BW-tween buffer.
7. Resuspend the Dynabeads in 20 ul 2× BW-tween buffer.
8. Mix the nuclease reaction together with 10 ul of resuspended Dynabeads
9. Incubate at room temperature with rotation for 30 minutes.
10. Wash the beads 3× with 1× BW-tween buffer, and 2× with H₂O. Rotate beads for 5 minutes during each wash, and transfer beads to a new tube after each wash.
11. Resuspend beads 6 ul H₂O to use as template in the amplicon adapter PCR.

Amplicon Adapter PCR and Purification:

For the adapter PCR, we suggest you use between ¼ and ½ of your total purified supernatant volume, to ensure that you have material remaining in case the reaction needs to be repeated.
Mix together the following PCR reagents:
   12.5 ul Kapa HiFi Hot Start Readymix
   0.75 ul 10 uM XT_Nested_F primer
   0.75 ul 10 uM Bead_R primer
   6 ul washed Dynabeads with amplicons bound
Run the following PCR:
   95° C. for 3:00
   For X cycles (optimized before experiment):
   98° C. for 0:10
   60° C. for 0:20
   72° C. for 0:30
   Then:
   72° C. for 3:00
   4° C. ∞
Add 25 ul of H₂O to bring the volume to 50 ul (this is to dilute the high salt concentration).
Purify the PCR using a zymo clean and concentrate column, as per the manufacturer's instructions.
Elute in 21 ul H₂O.
Load the eluent onto a lane of a 2% E-gel EX agarose gel. Run the gel as per the manufacturer's instructions. (Note: it is important to clean up the PCR before running it on the E-gel, as the high salt concentration of the Kapa mix will cause the gel to run inaccurately.)
Extract the intended amplicon band from the gel. The expected size of the amplicon should be calculated as follows:

Droplet PCR target size+30 bp polyA+45 bp bead oligo−nested primer depth+34 bp adapter. Actual band size may vary slightly based on polyA/polyT binding.
Purify the gel-extracted band using the NEB Monarch gel extraction kit, as per the manufacturer's instructions.
Elute in 15 ul H₂O.
Adapter PCR can be stored at 4° C. or −20° C.

Amplicon Indexing PCR and Purification:
Mix together the following PCR reagents:
   10 ul Kapa HiFi Hot Start Readymix
   0.5 ul 10 uM P5-SMART primer
   0.5 ul 10 uM N70× index primer
   3 ul purified adapter PCR
   6 ul H₂O
Run the following PCR:
   95° C. for 3:00
   For 15-20 cycles:
   98° C. for 0:10
   55° C. for 0:20
   72° C. for 0:30
   Then:
   72° C. for 3:00
   4° C. ∞
Purify the PCR using a Zymo clean and concentrate column, as per the manufacturer's instructions.
Elute in 23 ul H₂O for purification with E-gel SizeSelect 2% agaros gel, or 21 ul H₂O for purification with E-gel EX 2% agarose gel. If the amplicon is one single-sized band, purify using a SizeSelect gel. If the amplicon is a range of sizes (i.e. from a heterogeneous cell population with possible deletions or insertions), purify using an E-gel EX gel.
Follow the manufacturer's instructions for running the desired E-gel.
Extract or retrieve the intended amplicon from the gel. The expected size of the amplicon should be calculated as follows:

Adapter PCR amplicon size+41bp P5-SMART sequence+32bp N70×index sequence
Purify the amplicon using to Zymo clean and concentrate spin column if retrieved from a SizeSelect gel, or an NEB Monarch gel extraction spin column if extracted from an E-gel, as per the manufacturers' instructions.
Elute in 10 ul H₂O.
Assess the amplicon library on a BioAnalyzer High Sensitivity DNA chip, as per the manufacturer's instructions.
The amplicon library can be stored at 4° C. or −20° C.

Sequencing Your Libraries:

Libraries should be sequenced on a MiSeq for quality control and determining the number of cells, before moving on to deep sequencing. If you are sequencing cDNA libraries alone or in combination with the amplicon libraries, it is not necessary to spike in a PhiX control. If you are sequencing amplicon libraries alone, PhiX is required to introduce diversity into your library.

The Custom Read 1 Primer is required for sequencing the Drop-seq bead barcodes. If you are not using PhiX, the custom primer should be diluted and loaded as per Illumina's instructions: supportillumina.com/content/dam/illumina-support/documents/documentation/system_documentation/miseq/miseq-system-custom-primers-guide-15041638-01.pdf. When setting up your sequencing run parameters, you need to specify that custom read 1 primer is being used.

If using PhiX, then the custom primer should be diluted and loaded as per Illumina's primer spike-in instructions: support.illumina.com/bulletins/2016/04/spiking-custom-primers-into-the-illumina-sequencing-primers-.html. Otherwise, the PhiX will not be amplified during the run. It was determined that the Custom Read 1 Primer does not interfere with any of the Illumina sequencing primers when spiked in. When setting up the sequencing run parameters in this case, do not select the option for a custom read one primer.

Additional Sequencing Parameters:

Read 1:25 bp

Read 2: 100 bp (or more/enough to cover the regions of your amplicon sequence that needs to be to read for genotyping)

Read 1 index: 8 bp

Lastly, when determining the number of reads wanted, cDNA reads should be based on the number of cells, while amplicon reads should be based on the total number of beads.

### Example 3

This example illustrates analysis of mutated cell populations with DREAM-seq.

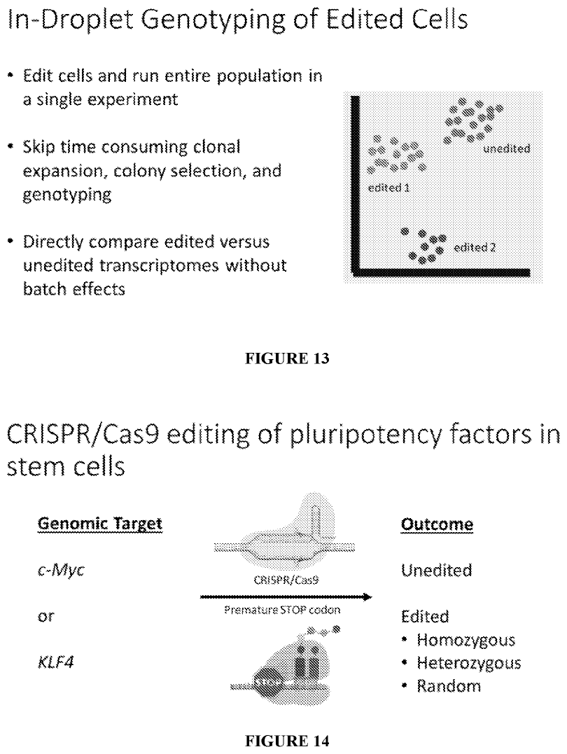

DREAM-seq was used for in-droplet genotyping of a population of cells with mixed mutational profiles. When CRISPR/Cas9 is used to introduce a genetic mutation into cells, the editing efficiency is less than 100% (FIG. 13). For example, cells after transfection can be homozygously or heterozygously edited, edited without the repair template, or not edited at all. CRISPR/Cas9 and microhomology-based repair templates were used to introduce a premature stop codon into either the c-Myc or KLF4 gene in a human iPS cell line (FIG. 14). Because each of these genes encodes for a transcription factor that is expressed to induce and maintain pluripotency in stem cells, interrupting their expression should introduce widespread and measurable changes in the transcriptome.

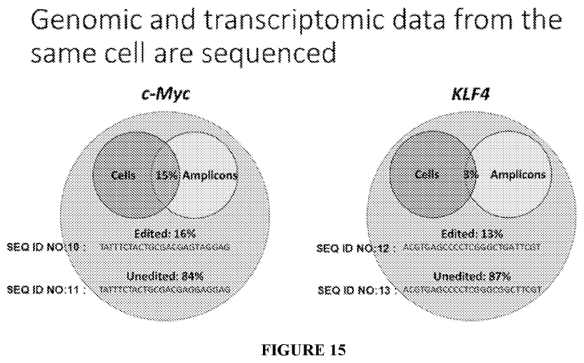

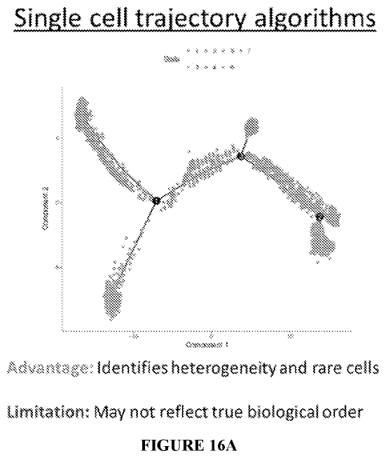

After transfection, entire cell populations were analyzed using DREAM-seq with primers to amplify respective target sites. After processing captured molecules into amplicon and cDNA libraries, libraries were sequenced on a Miseq and genotypes assigned to individual cells (FIG. 15). For both c-Myc and KLF4 experiments, a relatively consistent distribution was seen, with approximately 15% of amplicon reads showing edits in the sequence, and about 85% being wild type sequences (16% edited for c-Myc, 13% edited for KLF4; FIG. 15). Based on cDNA and shared barcodes, approximately 15% of cells were edited for c-Myc and approximately 3% of cells were edited for KLF4. Each cell barcode matched tens to hundreds of reads from the amplicon libraries, and several cells with both edited and unedited amplicon reads were seen as well, consistent with heterozyously edited cells. Without being limited by theory, further optimization may result in greater agreement between values obtained from amplicon and cDNA sequences.

These results show that direct in-droplet genotyping can be used for the identification of gene-edited cells, thereby bypassing a need for clonal expansion of potentially edited cells after transfection and recovery and repeated rounds of colony picking and genotyping that may take several months. Using primers that target potential edits, cells can be individually genotyped inside droplets, with amplicon sequences used to bioinformatically determine whether a cell is edited or unedited and what the edit is. Cells can then be clustered by identifying labels and gene expression can be compared directly between subpopulations.

### Example 4

This example illustrates use of simultaneous analysis of RNA and DNA from the same single cell for cell lineage tracing and DNA barcoding.

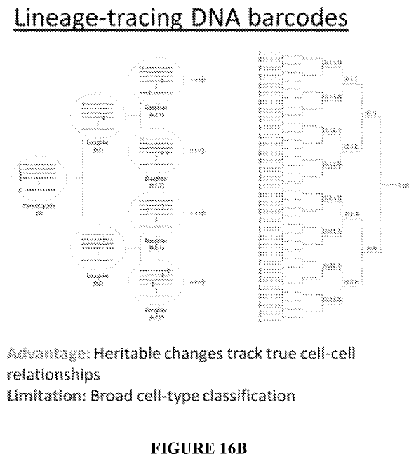

The DREAM-seq method for simultaneous analysis of RNA and DNA from the same single cell can be used to combine transcriptomics and lineage barcoding from single cells to analyze cell differentiation, for example. As shown in FIGS. 16A and 16B, single cell RNA-seq can be used to identify heterogeneity in a population of cells that can be observed over time, for example (FIG. 15A). Algorithms can then be used to predict cell trajectories based on gene expression patterns. As an example, cells from human stem cell-derived retinal organoids were arranged in pseudotime, with each dot representing a cell and each branch representing a different state of differentiation (FIG. 15A). Thus, cells can be distributed on a spectrum based on similarities and differences of gene expression patterns, with the optimal algorithm-produced tree representing a proxy for cell lineage that may not reflect the cells' true biological order because measurements are taken at different time points from different cells. While these measurements can be combined into an average approximation for how the transcriptome changes over time, individual cell development over time is not traced. Measurements are taken at different times from different cells if lineage barcodes were introduced, but the lineage is still being recorded in between the sampling points.

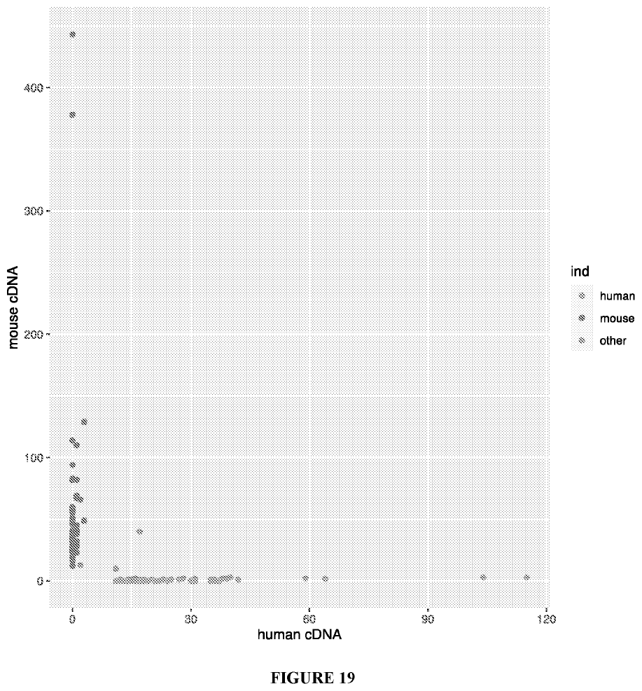

Editable DNA barcodes can be used to record and track cell relationships, reflecting a cell's true biological lineage (FIG. 19B). A DNA barcode sequence is inserted into cells that is subject to change, for example by recombinases or CRISPR/Cas9, allowing for the barcode and its changes through a cell and its daughters to be followed, as each cell barcode accumulates its own unique set of changes or scars. Lineage trees can be recreated by sequencing these DNA barcodes and comparing them to determine the degree of relatedness between cells. The limitation is that, in order to fully understand the potential of each progenitor cell, daughter cells need to be classified accurately. Currently, this classification is done with common cell type markers that can only distinguish broad populations of cells.

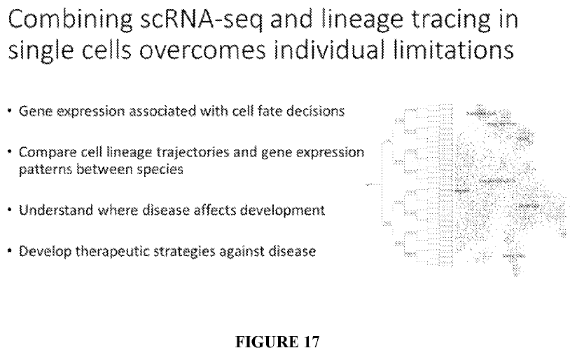

The individual limitations of scRNA-seq and lineage tracing in single cells can be overcome by combining the two techniques (FIG. 17). High-resolution maps of development can be created by merging cell type identification and gene expression analysis from scRNA-seq with lineage recording data from DNA barcodes, focusing on gene expression changes that are associated with specific lineage relationships. Without being limited by theory, because many diseases affect specific cell types, this kind of information can be used to determine what genes will reprogram a cell or act as targets for drug-based therapies, for example.

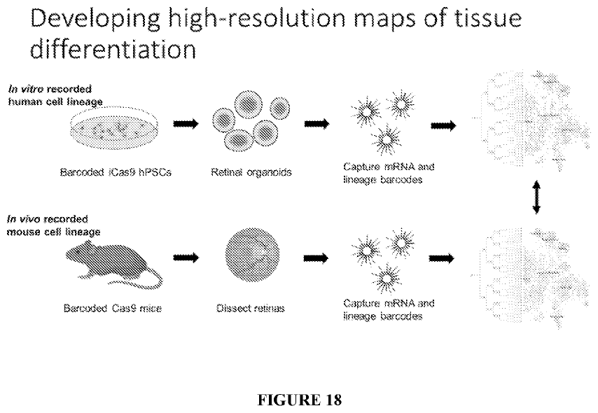

Tracing of cell lineages in retinal organoids from barcoded human stem cells or barcoded mice is shown in FIG. 18. Data from mouse model systems is complemented with data from retinal organoids derived from differentiated human cells. High resolution traces of transcriptomic changes that individual cells undergo throughout human retinal cell fate determination are created. Because a number of retinal diseases are attributed to developmental genes, high resolution maps are used to analyze where disease affects development. In addition, comparison of mouse and human retinal cell lineages is used to improve mouse studies and assess the extent of extrapolation of mouse data to human systems.

### Example 5

This example illustrates use of simultaneous analysis of RNA and DNA from the same single cell to distinguish cells from different species.

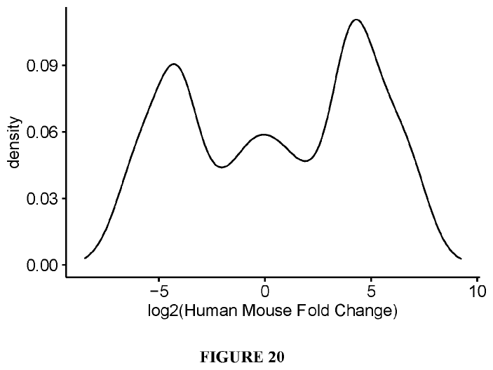

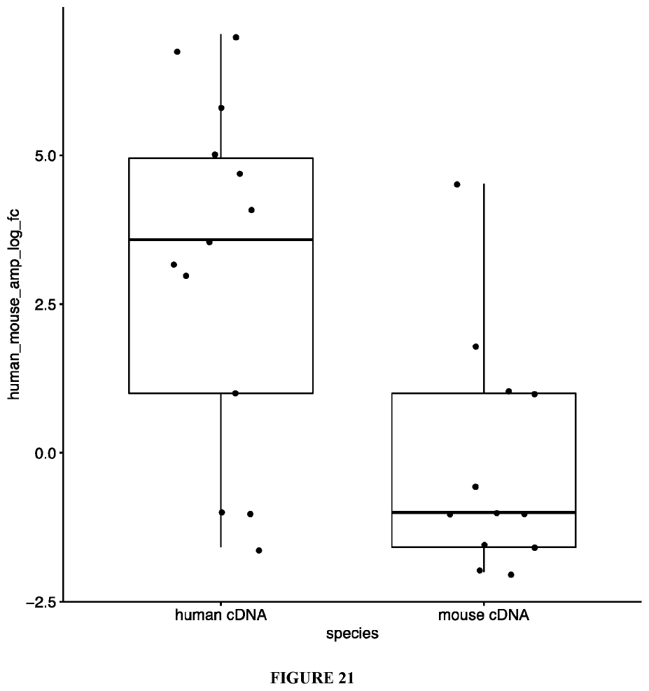

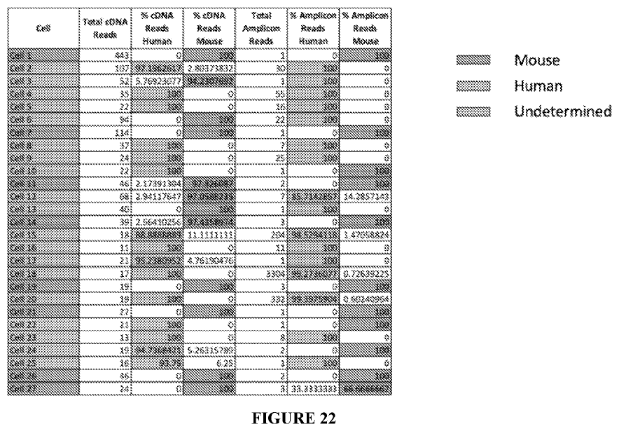

To validate the ability of DREAM-seq to characterize single cells simultaneously based on their genotype and transcriptomes, we performed species mixing using our method to target an intronic region of the PAX6 locus with a high degree of sequence conservation between mouse and human genomes. One pair of primers was designed to generate 180 base-pair amplicons from a single-cell suspension of equal parts human induced pluripotent cells and mouse Neuro-2a cells. Within the amplified region, three single nucleotide polymorphisms between mouse and human sequences were used to determine the species of the originating cell, as per the DNA readout. Based on transcriptomic alignment of the cDNA, 95% of cells were confidently identified as either human or mouse (FIG. 19). When analyzing the amplicon sequencing, cells were well divided by species, although a larger percentage of cells were determined as mixed, likely due to the inability to fully prevent template switching (FIG. 20).When comparing the DNA-based cellular identities to the species-specific alignments of the transcriptomes from each cell, we observed a visible correlation between the two types of data (FIG. 21). 74% of cells classified as human or mouse based on their transcriptome were identified as the same species based on their genomic amplicons (FIG. 22).

In summary, the above examples (Examples 1-5) show that DREAM-seq offers itself to a versatile set of applications, ranging from clinical diagnostics to developmental biology to high-throughput screens.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.1, 2.2, 2.7, 3, 4, 5, 5.5, 5.75, 5.8, 5.85, 5.9, 5.95, 5.99, and 6. This applies regardless of the breadth of the range.

Although the invention has been described with reference to the above examples, it will be understood that modifications and variations are encompassed within the spirit and scope of the invention. Accordingly, the invention is limited only by the following claims.

1. A method of simultaneously analyzing DNA and RNA from a same cell comprising:
(a) providing a droplet comprising a single cell, wherein the single cell is lysed providing nucleic acid in the droplet;
(b) performing a first polymerase chain reaction (PCR) reaction on DNA in the droplet, thereby generating amplicons comprising a 3′ poly(dA) sequence and a bead oligonucleotide sequence;
(c) capturing the RNA on a microparticle comprising a bead oligonucleotide sequence;
(d) breaking the droplet and separating the supernatant comprising the amplicon from the RNA captured on the microparticle; and
(e) performing a reverse transcription reaction transcribing the RNA including the bead oligonucleotide sequence.

2. The method of claim 1, further comprising preparing libraries of the separated amplicons and transcribed RNA and performing a PCR reaction on the transcribed RNA.

3. (canceled)

4. The method of claim 1, further comprising enzymatically modifying the amplicons using a lambda nuclease and a terminal transferase.

5. (canceled)

6. The method of claim 4, further comprising biotinylating the amplicons by biotinylated second strand synthesis and subjecting the amplicons to modification with mung bean nuclease, performing a second PCR reaction on the amplicons and performing a third PCR reaction on the amplicons.

7-8. (canceled)

9. The method of claim 6, wherein forward primers for the second and/or third PCR reactions include sites for sequencing.

10. The method of claim 1, further comprising sequencing the transcribed RNA molecules and enzymatically modified and amplified amplicons.

11. The method of claim 1, wherein the microparticle comprises a bead.

12. The method of claim 1, wherein the oligonucleotide sequences comprise a barcode comprising a cellular barcode and a unique molecular identifier.

13. (canceled)

14. The method of claim 12, wherein the oligonucleotide sequences further comprise a poly(dT) sequence and/or a PCR handle for reverse transcription and PCR.

15. (canceled)

16. The method of claim 1, further comprising mapping sequences of separated transcribed molecules comprising a matching cellular barcode to the same cell.

17-18. (canceled)

19. The method of claim 1, wherein first PCR reverse primers comprise a poly(dT) sequence a bead oligonucleotide sequence.

20. The method of claim 1, wherein the reverse transcription reaction comprises Moloney Murine Leukemia Virus (M-MLV)-reverse transcriptase and template switching oligonucleotides.

21. A method of simultaneously analyzing DNA and RNA from the same cell comprising:
(a) providing a droplet comprising a single cell, wherein the single cell is lysed providing nucleic acid in the droplet;
(b) performing a first polymerase chain reaction (PCR) reaction on DNA in the droplet, thereby generating amplicons comprising a 3′ poly(dA) sequence and a bead oligonucleotide sequence;
(c) capturing the RNA on a microparticle comprising a bead oligonucleotide sequence;
(d) breaking the droplets and separating the supernatant comprising the amplicons from the RNA captured on the microparticle;
(e) performing a reverse transcription reaction transcribing the RNA including the bead oligonucleotide sequence;
(f) enzymatically modifying the amplicons and performing a second PCR reaction on the modified amplicons; and
(g) performing a third PCR reaction on the transcribed RNA, thereby amplifying transcribed molecules.

22-38. (canceled)

39. A method of analyzing a transcriptome of a genome-edited cell comprising:
(a) determining a genotype of a single cell by sequencing transcribed amplicons prepared by the method of claim 1, thereby identifying edited and unedited cells;
(b) sequencing transcribed RNA prepared by the method of claim 1;
(c) mapping sequences of transcribed amplicons and sequences of transcribed RNA comprising a matching cellular barcode to the same cell; and
(d) grouping sequences of transcribed amplicons and sequences of transcribed RNA from edited and unedited cells according to matching genome edits.

40. (canceled)

41. The method of claim 39, wherein the single cells comprise a genomic barcode.

42. The method of claim 41, wherein edited cells comprise one or more mutations in the genomic barcode.

43-52. (canceled)

53. A method of determining tumor heterogeneity comprising simultaneously analyzing DNA and RNA from a tumor cell using the method of claim 1.

54. A method of determining somatic mosaicism comprising simultaneously analyzing DNA and RNA from a cell using the method of claim 1.

55-57. (canceled)

58. A method of screening for perturbations in cells modified with guide RNAs comprising simultaneously analyzing DNA and RNA of a cell in a population of modified cells using the method of claim 1.

59. The method of claim 58, wherein cells are modified using a library of guide RNAs representative of a range of genes.

60. The method of claim 58, wherein cells are modified using a gene modifying agent selected from the group consisting of a CRISPR-associated (Cas) protein, a Cre DNA recombinase, a TALEN, a zinc finger nuclease, a homing endonuclease, and a targeted SPO11 nuclease.

61. A method of probing genetic thresholds on a phenotype comprising simultaneously analyzing DNA and RNA from a cell using the method of claim 1.

62. (canceled)

63. A method of genotyping cells comprising simultaneously analyzing DNA and RNA from a cell using the method of claim 1.

64. (canceled)

65. A method of tracing a lineage of a cell comprising simultaneously analyzing DNA and RNA from the cell the lineage of which is being traced using the method of claim 1.

66-68. (canceled)

69. An oligonucleotide comprising a PCR handle for reverse transcription and PCR, a barcode, and a poly(dT) sequence.

70-72. (canceled)

73. The oligonucleotide of claim 69, further comprising captured DNA comprising a 3′ poly(dA) sequence, wherein the DNA is genomic DNA, mitochondrial DNA, or a combination thereof.

74-92. (canceled)
